(19) United States
(12) Patent Application Publication
(10) Pub. No.: US 2006/0154854 A1
(43) Pub. Date: Jul. 13, 2006

(54) Molecules for targeting and releasing therapeutic compounds, and the use thereof

(76) Inventors: Francoise Russo-Marie, Sevres (FR); Alain Samson, Gometz Le Chatel (FR)

Correspondence Address: NIXON & VANDERHYE, PC, 901 NORTH GLEBE ROAD, 11TH FLOOR, ARLINGTON, VA, 22203, US

(21) Appl. No.: 10/560,025

(22) PCT Filed: Jun. 9, 2004
(86) PCT No.: PCT/FR04/01435

§ 371 Date: Feb. 23, 2006

(30) Foreign Application Priority Data
Jun. 10, 2003 (FR) 03/06944

(51) Int. Cl. A61K 38/16

(52) U.S. Cl. 514/7; 514/12; 530/324

(57) ABSTRACT

The invention relates to chimera molecules for targeting and releasing therapeutic compounds in mammals, especially humans. The inventive molecules comprise essentially three functional segments or domains: a targeting segment that can preferentially bind to the surface of the targeted cells, a therapeutic segment comprising the biologically active compound, and a linker segment between the targeting segment and the therapeutic segment, the linker segment being cleavable onto the target site. The invention also relates to the preparation of said molecules, to synthesis intermediates or domains thereof, to pharmaceutical compositions containing the same, and to the use thereof, especially in the pharmaceutical field. The molecules and compositions according to the invention are especially adapted to the targeting of pathological cells engaged in an apoptotic phase, and to the treatment of pathologies or associated tissues, especially cancers and inflammation.

### TECHNICAL FIELD

This invention relates to chimera molecules for targeting and releasing therapeutic compounds in mammals, especially humans. The molecules according to the invention have essentially three functional segments or domains: a targeting segment, that can preferentially bind to the surface of the targeted cells, a therapeutic segment comprising the biologically active compound, and a linker segment between the targeting segment and the therapeutic segment, the linker segment being cleavable onto the target site. The invention also relates to the preparation of said molecules, to synthesis intermediates or domains thereof, to pharmaceutical compositions containing the same, and to the use thereof, especially in the pharmaceutical field. The molecules and compositions according to the invention are especially adapted to the targeting of pathological cells in an apoptotic phase, and to the treatment of pathologies or associated tissues, especially cancers and inflammation.

### BACKGROUND

In principle, anti-tumoral compounds are highly cytotoxic compounds which are distributed over the whole of the organism when they are administered systemically. They produce secondary reactions which are extremely prejudicial to the individual. It is therefore greatly beneficial to only distribute these anti-tumoral compounds in the tumoral tissues, i.e. to target these tissues by means of a specific vector. Although this problem relating to the toxicity of treatments is particularly acute for cancer, it can apply to a large number of treatments in which, in general, a minimum secondary effect is sought for maximal therapeutic effect.

Different vectors have been proposed, and in particular monoclonal antibodies directed against markers specific of the surface of cancerous cells. In fact they produce cytotoxic effects on the tumoral cells, but the amplitude of these effects can diminish over time.

A system called ADEPT (Antibody Directed Enzyme Prodrug Therapy) consists of a therapy wherein an antibody conveys an enzyme to the tumoral site. Then, a prodrug is administered and converted into an active molecule by this enzyme (U.S. Pat. No. 5,760,072; U.S. Pat. No. 5,433,955). In this method, only the enzyme enabling activation of the prodrug is the object of targeting. The prodrug is therefore distributed over the whole of the organism, which does not entirely exclude secondary reactions and reduces the dose of prodrug effectively supplied to the tumoral site. Furthermore, this approach is complex because it requires the use of several types of molecule. A similar method based upon the targeting of the enzyme capable of activating the prodrug was described in applications (WO97/26918; WO 98/51787).

On the other hand, various binding systems for the production of prodrugs, with the possibility of targeting, have been described: for example, one can find sulfonamide bonds (WO98/00173), bonds which are cleavable by cathepsin B (WO98/56425) and cinnamate bonds (US20020187992).

However, there is still a great need for non-toxic therapeutic molecules or approaches enabling targeting and specific activation of a prodrug at a pathological site. This system would make it possible to reduce the dose of active agent, to improve effectiveness, and to reduce the secondary effects.

### DESCRIPTION OF THE INVENTION

The object of the present invention is to provide molecules capable of targeting tissues with a pathology and to specifically release compounds there with therapeutic properties for this pathology. By means of the molecules of the invention, the therapeutic compounds are active locally and more effectively, and offer greatly reduced risk of secondary effects.

The molecules of the invention can be designed to target different types of cells or pathological tissues, preferably in human. In a preferred embodiment, the invention is based on the targeting of apoptotic cells, by means of a targeting element with the property of binding to cellular membranes expressing a negatively charged lipid, in particular phosphatidylserine (PS). Apoptosis, or programmed cell death, is a normal physiological phenomenon, characteristic of multicellular organisms and present in particular in human. Under normal conditions, the apoptosis rate is weak however and the apoptotic cells are very quickly phagocyted either by the neighbouring cells, or most frequently by the professional phagocyte cells such as macrophages or dendritic cells. Furthermore, the apoptotic sites are often highly dispersed and do not show any synchronisation. For this reason, physiological apoptosis is generally undetectable.

Apoptosis is signalled to the phagocyte cells by the presence of PS on the surface of the apoptotic cells, resulting from the loss of asymmetry of their plasmic membrane. The presence of detectable apoptosis is the sign of a physiological disorder where the phagocyte functions are extended and incapable of coping with the increase in cells to be eliminated. Well known examples of this type of disorder are for example apoptosis of the cardiac muscle following an infarct or hepatic apoptosis following a strong viral infection or other affections with an acute character. Apoptosis cells are also present in other tissues or pathological mechanisms such as inflammation and cancer.

On the other hand, when the pathological tissue contains little or not sufficient target cells capable of retaining a large number of the therapeutic molecules of the invention, the latter can be used in combination with an agent or a treatment causing or favouring apoptosis within the pathological tissue so as to increase the therapeutic benefit. For example, some “young” cancerous tissue (e.g., small in size or non-metastasized) do not always contain a significant quantity of apoptotic cells, taking into account their rapid division. In this case, an agent favouring apoptosis (for example a classic anti-tumoral agent) can be used in combination with the molecules of the invention, at least at the starting of the treatment, so as to increase the therapeutic effectiveness.

The advantage of targeting the apoptotic cells of the tumoral environment, rather than targeting specific markers on the surface of the tumoral cells (for example, by means of monoclonal antibodies), is the kinetic effect linked to administration of the anti-tumoral compound: in the first case, the action of the therapeutic compound, if it is administered continuously, produces an increase in the “target” and so in the effective concentration of said therapeutic compound in the tumoral environment whereas, in the second case, the size of the “target” tends to reduce as does the effective concentration of the therapeutic compound. This cumulative effect resulting from the targeting of the apoptotic cells is also very important for controlling the initial metastasis steps by maintaining an optimal concentration of the therapeutic compound in the final phases of the tumour regression. Of course, the strong reduction in the effective dose of anti-tumoral compound and its strong location in the tumoral region also has the effect of considerably reducing the secondary toxic effects upon the patient.

Therefore, the molecules of the invention make it possible to target different pathological tissues and to release there a therapeutic or biologically active agent in situ, thus reducing their undesirable cytotoxic effects upon the healthy tissues.

A first object of the invention is therefore chimera molecules having a targeting region, an active region and a linker region sensitive to the environment of a tissue or of a pathological cell. The targeting region is preferably a region targeting the apoptotic cells. The active region can be any therapeutic compound, typically anti-cancerous or anti-inflammatory. The therapeutic compound is typically less active when it is in the form of a chimera molecule of the invention, than when it is in a free form.

Another object of the invention concerns a pharmaceutical composition including a chimera molecule as defined above.

Another object of the invention concerns the use of the targeting molecules as defined above for preparing medications. In a preferred embodiment, these medications are anti-tumoral or anti-inflammatory medications. The invention can be used in particular for the treatment of solid or liquid or hematopoietic tumours, in particular of cancers of the breast, lung, intestine, colon, prostate, brain, head and neck, liver, skin, lymphoma, melanoma, etc. When the therapeutic compound is an anti-inflammatory, the molecules according to the invention can be used for preparing medications intended for the treatment of acute or chronic inflammatory diseases such as asthma, hemorrhagic rectocolitis, Crohn's disease, septic shock, collagen diseases and arthritis.

Another object of the invention concerns the use of targeting molecules as defined above for the local supply of active principles around the pathological tissues in patients.

Furthermore, this invention concerns methods for treating a disease in a subject comprising administration of a molecule or of a composition as defined above. Preferably, said disease is a cancer or an inflammation. The treatment method can further include a preliminary step consisting of a treatment allowing to generate cells engaged in an apoptosis process in the pathological tissue. It also concerns methods for locally supplying active principles to the surrounding of pathological tissues in subjects, comprising administration of a molecule or of a composition as defined above.

These different aspects of the invention will be described in greater detail in the following text.

Molecule for Targeting and Releasing Therapeutic Compounds

As indicated above, the molecules of the invention typically comprise three parts or functional domains bonded to one another, namely a targeting region (C), a biologically active region (A) and a linker region (L) sensitive to the environment of a tissue or of a pathological cell. The arrangement of the different elements can vary, and in particular according to the order A-L-C or C-L-A. On the other hand, in some embodiments, the region (or the function) L can be inserted or included within either of the regions A or C.

More preferably:
1) The targeting segment C is a molecule capable of recognising or binding preferably cells involved in a pathological process, preferably apoptotic cells;
2) The linker segment L is a molecule ensuring binding between A and C, said linker segment being cleavable on the target site enabling release of the therapeutic segment A;
3) Part A is a biologically active molecule with therapeutic properties.
   Targeting Segment C

The targeting segment C includes a polypeptide capable of binding to the surface of cells which are present in a characteristic or specific way in a tissue having a pathology, or generated in this tissue by a preliminary or combined treatment. This targeting segment C is preferably capable of binding to the membranes of the cells engaged in an apoptosis process, these cells displaying on their surface negatively charged lipids such as phosphatidylserine.

In a first preferred embodiment, part C comprises a polypeptide capable of binding preferably to the surface of tumoral cells or those present in a tumoral tissue or generated in these tissues by treatment by means of an agent favouring apoptosis, for example an anti-tumoral agent (chemotherapy, radiotherapy etc.).

In another preferred embodiment, the targeting segment C comprises a polypeptide capable of binding preferably to the surface of the cells present in an inflammatory tissue and in particular the neutrophilic cells which accumulate in these tissues and die there by apoptosis 24 to 48 hours after their arrival. The neutrophils provide “bait” for the targeting molecules.

The term “binding preferably” indicates that the targeting element has a particular affinity for the cells or tissues being considered, even if non-specific or less important binding with other cells or tissues can not be totally excluded in vivo. The preferred binding guarantees, nevertheless, targeting of the chimera molecules of the invention to the pathological sites, reducing dissemination and the potential secondary effects.

Targeting segment C is preferably a peptidic molecule. Peptidic molecule refers to any molecule consisting of or including amino acids, natural or not, possibly modified, such as for example any protein or fragment of protein, a polypeptide or peptide, natural or synthetic, modified or not. The common property of these elements is to be able to bind preferably to cells characteristic of pathological situations and, in a preferred embodiment, to the cellular membranes displaying negatively charged lipids, in particular phosphatidylserine. In general, the present invention proposes the use of any protein, fragment or derivative of a protein meeting this criterion.

Several families of protein which are capable of binding to the membranes displaying negatively charged lipids exist. One can cite in particular the Annexin family, the families of proteins comprising a C1 or C2 domain, such as factors V and VIII of blood coagulation; the families of proteins comprising a PH domain or a FYVE domain; or else proteins comprising a domain identical or similar to domain 5 of the β2-Glycoproteins-I (βGP-I). These proteins, or domains originating from or derived from their sequences can be used as a targeting element in the chimera molecules of the invention. For reasons relating to immunogenicity, one preferably chooses the human version of these proteins or protein domains. Moreover, every time that it is possible it is appropriate to select and use the smallest active domain of these proteins so as to guarantee the best diffusion of the molecule through microvascularisation to the targeted tissues and in particular better bio-distribution and elimination via the renal route.

In a particular embodiment of this invention, the targeting element comprises a peptidic sequence derived from the proteins or fragments of proteins mentioned above. Fragment refers to a sequence of at least 10 consecutive amino acids, preferably between 50 and 500 amino acids, and even more preferably between approximately 250 and 350 amino acids. These derivatives have at least 50% of identity with the initial proteins or the fragments thereof. Preferably, they have 60%, 75%, 90% or 95% of identity. Some of the protein domains mentioned, in particular the PH and FYVE domains, can moreover be mutated such as to modify their lipidic specificity so as to adapt them to the precise needs of the targeting of cells during the apoptotic phase.

Targeting segment C can contain one or more binding sites to segment L. The presence of several sites offers the advantage of being able to distribute several therapeutic molecules A at one time and to increase in the same proportions the concentration of A in the environment of the tissues effected by the pathology in question.

In a particular embodiment, the structure of the molecule according to this invention is therefore:
C-(L-A)ₙ or (A-L)ₙ-C where n≧1 (F1).

In another embodiment, which can possibly be combined with the previous one, targeting segment C can comprise repetition of several polypeptides or motifs binding to the pathological cells or targets (referred to as C0), so as to increase effectiveness of the targeting. The general formula for these molecules is as follows:
(C0)ₘ-(L-A)ₙ or (A-L)ₙ-(C0)ₘ (F2)
where n and m are, independently of one another, an integer greater than or equal to 1.

In order to optimise the in vivo behaviour of the therapeutic molecule, one preferably chooses m and/or n=1 or 2.

According to a first specific variation of the invention, targeting segment C comprises the sequence of an annexin, or of a fragment or a derivative thereof. Targeting segment C preferably comprises the “four-domain core” sequence of a protein of the family of annexins. Preferably, the annexin is type V, a fragment or a derivative thereof. Human annexin is favoured. Preferably, targeting segment C comprises domain 1 of this annexin.

According to a first specific variation of the invention, targeting segment C comprises a type C1 or C2 segment, a fragment or a derivative thereof. More particularly, the invention relates to targeting segments C comprising the sequence of a C1 domain of a coagulation factor, a fragment or a derivative thereof. Alternatively, the invention concerns targeting segments C comprising the sequence of a C2 domain of the human coagulation factor VIII, a fragment or a derivative thereof.

In a preferred embodiment, targeting segment C is a polypeptide designed on the basis of a C1 type domain topology. Preferably, targeting segment C comprises a polypeptide of a sequence selected from SEQ ID Nos 1-8, preferably SEQ ID Nos 2-4, and 6-8, or a fragment thereof.

Type C1 Domain of Human Coagulation Factor V-CIF5-S0 (F-V) Wildtype Sequence

(SEQ ID No 1)

DCRMPMGLST GIISDSQIKA SEFLGYWEPR LARLNNGGSY
NAWSVEKLAA EFASKPWIQV DMQKEVIITG IQTQGAKHYL
KSCYTTEFYV AYSSNQINWQ IFKGNSTRNV MYFNGNSDAS
TIKENQFDPP IVARYIRISP TRAYNRPTLR LELQGC

Polypeptides Designed on the Basis of the C1 Type Domain of Human Coagulation Factor V

C1F5-S1

(SEQ ID No 2)

DCRMPLGMST GIISDSQIKA SEFLGYWEPR LARLNNGGSY
NAWSVEKLAA EFASKPWLQI DMQKEVIITG IQTQGAKHYL
KSCYTTEFYI AYSSNQINWQ IFKGNSTRNV MYFNGNSDAS
TIKENQLDPP IVARYIRISP TRAYNRPTLR LELQGC
C1F5-S2

(SEQ ID No 3)

DCRMPMGLST GIISDSQIKA SEFLGYWWPR LARLNNGGSY
NAWSVEKLAA EFASKPWIQV DLQKEVIITG IQTQGAKHYL
KSCYVTEFYV AYSSNQINWQ IFKYNSTRNV MYFNGNSDAS
TIKENQFDPP LVARYIRISP TRAYNRITLR LELQGC
C1F5-S3

(SEQ ID No 4)

DCRMPMGLST GIISDSQIKA SEFLGYWEPR LARLNNGGSY
NAWSVEKLAA EFASKPWLQI DLQKEVIITG IQTQGAKHYL
KSCYTTEFYI AYSSNQINWQ IFKGNSTRNV MYFNGNSDAS
TIKENQLDPP IVARYIRISP TRAYNRPTLR LELQGC

C1 Type Domain of Human Coagulation Factor VIII-C1F8-S0 (F-V) Wildtype Sequence

(SEQ ID No 5)

KCQTPLGMAS GHIRDFQITA SGQYGQWAPK LARLHYSGSI
NAWSTKEPFS WIKVDLLAPM IIHGIKTQGA RQKFSSLYIS
QFIIMYSLDG KKWQTYRGNS TGTLMVFFGN VDSSGIKHNI
FNPPIIARYI RLHPTHYSIR STLRMELMGC

Polypeptides Designed on the Basis of the C1 Type Domain of Human Coagulation Factor VIII

C1F8-S1

(SEQ ID No 6)

KCQTPMGLAS GHIRDFQITA SGQYGQWAPK LARLHYSGSI
NAWSTKEPFS WLKIDLLAPM IIHGIKTQGA RQKFSSLYIS
QYIIMYSLDG KKWQTYRGNS TGTLMVFFGN VDSSGIKHNI
FNPPIIARYI RLHPTHYSIR STLRMELMGC
C1F8-S2

(SEQ ID No 7)

KCQTPMGLAS GHIRDFQITA SGQYGQWAPK LARLHYSGSI
NAWSTKEPFS WIKVDLLAPM IIHGVKTQGA RQKFSSLYIS
QFIIMYSLDG KKWQTYRYNS TGTLMVFFGN VDSSGIKHNI
FNPPLIARYI RLHPTHYSIR STLRMELMGC
C1F8-S3

(SEQ ID No 8)

KCQTPLGMAS GHIRDFQITA SGQYGQWWPK LARLHYSGSI
NAWSTKEPFS WLKIDLLAPM IIHGIKTQGA RQKFSSLYIS
QFIIMYSLDG KKWQTYRGNS TGTLMVFFGN VDSSGIKHNI
FNPPLLARYI RLHPTHYSIR STLRMEVMGC

In another preferred embodiment, targeting segment C is a polypeptide designed on the basis of a C2 type domain topology. Preferably, targeting segment C comprises a polypeptide of a sequence selected from SEQ ID Nos 9-16, preferably SEQ ID Nos 10-12, and 14-16, or a fragment thereof.

C2 Type Domain of Human Coagulation Factor V-C2F5-S0 (F-V) Wildtype Sequence

(SEQ ID No 9)

CSTPLGMENG KIENKQITAS SFKKSWWGDY WEPFRARLNA
QGRVNAWQAK ANNNKQWLEI DLLKIKKITA IITQGCKSLS
SEMYVKSYTI HYSEQGVEWK PYRLKSSMVD KIFEGNTNTK
GHVKNFFNPP IISRFIRVIP KTWNQSITLR LELFGCDIY

Polypeptides Formed Baseed on the C2 Type Domain of Human Coagulation Factor V

C2F5-S1

(SEQ ID No 10)

CSTPLGMENG KIENKQITAS SFKKSWWGDY WEPFRARLNA
QGRVNAWQPK ANNNKQWLEV DLLKIKKITA VITQGCKSLS
SEMYVKSFTI HYSEQGVEWK PFRLKSSMVD KINEGNTNTK
GHVKNFPNPP RISRFIRVIP KTWNQSITLR LELFGCDIY
C2F5-S2

(SEQ ID No 11)

CSTPLGIENG KIENKQITAS SFKKSWWGDY WEPFRARLNA
QGRVNAWQAK ANNNKQWLEM DFLKIKKVTA VITQGCKSLS
SEMYVKSFTI HYSEQGVEWK PYRLKSSMVD KIFEGNTNTK
GHVKNFFNPP IISRFIRQIP KTWNQSITLR LELYGCDIY
C2F5-S3

(SEQ ID No 12)

CSTPLGIENG KIENKQITAS SFKKSWWGDY WEPFRLRLNA
QGRVNAWQAK ANNNKQWAEM DLLKIKKITA IITQGCKSLS
SEMYVKSYTI HYSEQGVEWK PYRLKSSMVD KIFEGNTNTK
GHVKNFFNPP IITRFIRVIP KTWNQSITIR LELFGCDIY

C2 Type Domain of Human Coagulation Factor VIII-C2F8-S0 (F-V) Wildtype Sequence

(SEQ ID No 13)

CSMPLGMESK AISDAQITAS SYFTNMFATW SPSKARLHLQ
GRSNAWRPQV NNPKEWLQVD FQKTMKVTGV TTQGVKSLLT
SMYVKEFLIS SSQDGHQWTL FFQNGKVKVF QGNQDSFTPV
VNSLDPPLLT RYLRIHPQSW VHQIALRMEV LGC

Polypeptides Designed on the Basis of the C2 Type Domain of Human Coagulation Factor VIII

C2F8-S1 (SEQ ID No 14)
CSMPLGMESK AISDAQITAS SYFTNMFATW SPSKARLHLQ GRSNAWRAQV
NNPKEWLQID LQKTMKITGI TTQGVKSLLT SMYVKEYLIS SSQDGHQWTL
FYQNGKVKVF QGNQDSFTPV VNSLDPFLLT RYLRIHPVSW VHQIALRMEV
LGC
C2F8-S2 (SEQ ID No 15)
CSMPLGMESK AISDAQITAS SYKTNMFATW SPSKARLHLQ GRSNAWRAQV
NNPKQWLQVD FQKTMKVTGV TTQGVKSLLT SMYVKEFLIS SSQDGHQWTL
FFQNGKVKVF QGFQDSFTPV VNSLDPPLLT IYLRIHPQSW VHQIALRMEV
LEC
C2F8-S3 (SEQ ID No 16)
CSMPLGMESK AISDAQITAS SYKTNMFATW SPSKARLHLQ GRSNAWRPQV
NNPKEWLQVD FQKTMKVTGV TTQGVKSLLT SMYVKEYLIS SSQDGHQWTL
FYQNGKVKVF QGNQDSFTPV VNSLDPFLLT RYLRIHPQSW VHQIALRMEV
LEC

C2F8-S1

(SEQ ID No 14)

CSMPLGMESK AISDAQITAS SYFTNMFATW SPSKARLHLQ
GRSNAWRAQV NNPKEWLQID LQKTMKITGI TTQGVKSLLT
SMYVKEYLIS SSQDGHQWTL FYQNGKVKVF QGNQDSFTPV
VNSLDPFLLT RYLRIHPVSW VHQIALRMEV LGC
C2F8-S2

(SEQ ID No 15)

CSMPLGMESK AISDAQITAS SYKTNMFATW SPSKARLHLQ
GRSNAWRAQV NNPKQWLQVD FQKTMKVTGV TTQGVKSLLT
SMYVKEFLIS SSQDGHQWTL FFQNGKVKVF QGFQDSFTPV
VNSLDPPLLT IYLRIHPQSW VHQIALRMEV LEC
C2F8-S3

(SEQ ID No 16)

CSMPLGMESK AISDAQITAS SYKTNMFATW SPSKARLHLQ
GRSNAWRPQV NNPKEWLQVD FQKTMKVTGV TTQGVKSLLT
SMYVKEYLIS SSQDGHQWTL FYQNGKVKVF QGNQDSFTPV
VNSLDPFLLT RYLRIHPQSW VHQIALRMEV LEC

In a preferred embodiment, targeting segment C is a polypeptide designed on the basis of a domain 5 type topology of the β2-Glycoproteins-I (β2GP-I) (SEQ ID No 17). Preferably, targeting segment C comprises a polypeptide of a sequence selected from SEQ ID Nos 17-22, preferably SEQ ID Nos 18-22, or a fragment thereof.

Domain 5 of the Human 132-Glycoproteins-I-β2GP-I Wildtype Sequence

(SEQ ID No 17)

TKASCKVPVK KATVVYQGER VKIQEKFKNG MLHGDKVSFF
CKNKEKKCSY TEDAQCIDGT IEVPKCFKEH SSLAFWKTDA
SDVKPC

In a preferred embodiment, targeting segment C comprises a polypeptide having the following general sequence:

T J₂A S C K U₇ P U₉ K J₁₁ U₁₂ T U₁₄ U₁₅ U₁₆ J₁₇ G E R U₂₁ J₂₂ U₂₃ Q E K U₂₇ J₂₈ N G M L H G D K U₃₇ S F U₄₀ C J₄₂ N J₄₄ E J₄₆ J₄₇ C J₄₉ Y T E D U₅₄ Q C I D G T U₆₁ E V P K C U₆₇ J₆₈ E H S J₇₂ U₇₃ U₇₄ J₇₅ J₇₆ J₇₇ T D A S D V J₈₄ P C (SEQ ID No 18) (S4)

wherein:

J2=K, D, E; J11, J22, J28, J42, J44, J46, J47, J68, J77, J17=Q, E; J84=K, R; J49=S, T; J72=S, T, M; U7=L, V, I; U9=V, I, T; U12=A, M; U14, U15, U21, U23, U37=V, I, T; U16, U27, U40, U67=F, Y; U54=A, V, I; U61=I, V, M; U73, U74, U75, U76=L, I, F, Y, M, W.

Preferably, targeting segment C comprises a polypeptide of a sequence selected from SEQ ID Nos 19-22 or a fragment thereof:

Polypeptides Designed on the Basis of Domain 5 of the Human β2-Glycoproteins-I

GPI-S1

(SEQ ID No 19)

TEASCKVPVK RATVVYEGER VRIQEKFKNG MLHGDKVSFF
CRNRERRCSY TEDAQCIDGT IEVPKCYREH SMLTWWRTDA
SDVKPC
GPI-S2

(SEQ ID No 20)

TEASCKLPTK RMTVVYEGER VRIQEKFKNG MLHGDKISFF
CRNRERRCSY TEDAQCIDGT IEVPKCYREH SMITWWRTDA
SDVKPC
GPI-S3

(SEQ ID No 21)

TKASCKVPTK KMTVVYQGER VKIQEKFKNG MLHGDKISFF
CKNKEKKCSY TEDAQCIDGT IEVPKCYKEH SSLAWWKTDA
SDVKPC
GPI-S4

(SEQ ID No 22)

TKASCKVPTK KMTVVYQGER VKIQEKFKNG MLHGDKISFF
CKNKEKKCSY TEDAQCIDGT IEVPKCYKEH SSLAFWKTDA
SDVKPC

In a preferred embodiment, targeting segment C comprises a polypeptide having the following general sequence:

J¹-J²-J³-J⁴-J⁵-J⁶-Z⁷-U⁸-J⁹-J¹⁰-U¹¹-R-J¹³-J¹⁴-U¹⁵-k-g-X¹⁸-G-T-J²¹-E-J²³-J²⁴-U²⁵-J²⁶-J²⁷-H²⁸-U²⁹-J³⁰-J³¹-R-J³³-J³⁴-J³⁵-J³⁶-B³⁷-J³⁸-J³⁹-U⁴⁰-J⁴¹-J⁴²-J⁴³-U⁴⁴-J⁴⁵-J⁴⁶-J⁴⁷-J⁴⁸-J⁴⁹-R-J⁵¹-U⁵²-J⁵³-J⁵⁴-D-U⁵⁶-K-S-Z⁵⁹-L-J⁶¹-J⁶²-J⁶³-J⁶⁴-Z⁶⁵-J⁶⁶-J⁶⁷-U⁶⁸-J₆₉-J⁷⁰-J⁷¹-U⁷²-J⁷³-J⁷⁴-J⁷⁵-J⁷⁶
(S5)
wherein J, Z, U, X, and B represent amino acids such as:
   the J amino acids are selected independently of one another from natural amino acids, or from derivatives of the same, such that at least 50% of them are polar residues selected from R, N, D, C, Q, E, G, H, K, Orn, P, S, T and Y,
   the U amino acids are selected from A, C, G, I, L, M, F, W, Y, and V,
   amino acid X¹⁸ is selected independently of the other amino acids of the sequence from A, N, C, Q, G, H, I, L, M, F, S, T, W, Y and V,
   amino acid B³⁷ is selected independently of the other amino acids of the sequence from R, A, C, G, I, L, M, F, W, Y, and V,
   amino acid Z⁷ is selected independently of the other amino acids of the sequence from D and E,
   amino acids Z⁵⁹ and Z⁶⁵ are selected independently from E, D, K, and R,
the exponents indicating the position of the amino acids in the sequence.

Preferably, the J amino acids can be selected independently of one another from all of the residues A, R, N, D, C, Q, E, G, H, I, L, K, M, Orn, F, P, S, T, W, Y, and V, and such that at least 50% of them are polar residues selected from R, N, D, C, Q, E, G, H, K, Orn, P, S, T.

Different combinations of residues U and B are given in table 1 below:

TABLE 1

| | U⁸ | U¹¹ | U¹⁵ | U²⁵ | U²⁹ | B³⁷ | U⁴⁰ | U⁴⁴ | U⁵² | U⁵⁶ | U⁶⁸ | U⁷² |
|---|---|---|---|---|---|---|---|---|---|---|---|---|
| Ex 1 | V | L | M | I | L | R | I | Y | L | L | V | L |
| Ex 2 | A | I | I | I | L | R | I | Y | L | L | I | L |
| Ex 3 | A | I | I | I | L | R | I | Y | L | L | M | V |
| Ex 4 | A | L | M | L | L | R | I | Y | L | L | I | M |
| Ex 5 | A | L | M | I | I | R | V | Y | L | L | I | M |
| Ex 6 | A | L | M | I | I | R | I | F | L | L | I | M |
| Ex 7 | A | L | M | I | V | R | I | F | L | L | I | F |
| Ex 8 | V | L | M | I | L | R | I | F | L | L | I | M |
| Ex 9 | A | L | M | I | L | R | I | F | L | L | I | M |
| Ex 10 | A | L | M | I | L | R | I | Y | L | L | A | A |
| Ex 11 | V | L | M | I | L | R | I | Y | L | L | V | L |
| Ex 12 | V | L | M | I | L | R | I | F | L | L | V | L |

As an example, the peptide of formula S1 can advantageously be a peptidic sequence selected from the peptidic sequences SEQ ID No 23-32.

Sequence S1 represents a type of peptides in its shortest form. Of course this sequence can further comprise one or more additional amino acids at one or other of the ends, for example between 1 and 15 amino acids, in general between 1 and 10 amino acids in order to obtain additional functionalisation. For example, a small sequence, called a functionalisation sequence, can be bonded to the peptide enabling fixing to segment L. This functionalisation sequence can be located at the N-terminal end of the S1 sequence. It can be approximately 3 amino acids preferably selected from G-S-C-, G-S-T-, G-S-P-, G-S-S-, G-S-G-, and G-S-Q-. It can also be approximately 4 amino acids, preferably selected from sequences G-S-Aa-C-, G-C-Aa-S-, G-S-Aa-S-, G-C-Aa-C-, and G-C-Aa-S- where Aa is any amino acid.

These functionalisation sequences are advantageous in that they make it possible in particular an easy labelling using radioactive-tracers such as ^{99m}Tc or ¹⁸F and make it possible, by injecting into the man a tracer dose, to follow perfectly in vivo the progress of the medication, its biodistribution and to control its correct location.

Moreover, sequence S1 can be duplicated within a same peptide in order to produce a molecule with an even greater affinity to the apoptotic sites.

Linker Segment L

Segment L is a linker molecule cleavable onto the target site, and linking part A and part C. Segment L can be any molecule or chemical bond, of a covalent type, including molecules which are partially or fully peptidic in nature, modified or not, natural or not. Segment L advantageously contains a recognised chemical function which can be cleaved in the environment of the tissue or the pathological cells, for example by an enzyme or a set of enzymes specific of the environment of the targeted cells.

The presence, between parts A and C, of the linker segment L which can be cleaved preferably in the environment of the targeted cells makes possible local and targeted release of the active principle and attributes targeted prodrug type behaviour to the molecules of the invention. Indeed, the molecule is only slightly active when not located in the environment of the targeted cells having the enzymes or other cleaving factors.

Linker segment L can be single or branched. The benefit of branching is to be able to convey and distribute several therapeutic molecules with a single targeting molecule C. In this embodiment, the general structure of the linker segment is therefore L=D(L0)ₙ, where D is a branching element, n is an integer equal to or greater than 1 (corresponding to the number of arms of the ramification), and L0 is the actual linker.

As a result of this, the compound which is the object of this invention has one of the two following general formulae according to the order in which the different parts come:
C-D-(L0-A)ₙ (F4A)
(A-L0)ₙ-D-C (F4B)

Segment L (or L0) can be varied in nature, like a linker or chemical molecule, and in particular peptidic in nature.

In a preferred embodiment, segment L is a peptidic cleavable linker sensitive to proteases (or other enzymes), called in the following text “intervening proteases”, more specifically over-expressed either on the surface of the cells with the pathology, or released into the environment of the targeted cells, or else released by the cells in apoptotic phase.

Tumoral and inflammatory cells, as well as, for example, the stromal cells recruited in the area surrounding the latter, are known to excrete a variety of intervening proteases, in particular metalloproteases of the extracellular matrix (MMP), urokinases, proteases specific to the cleavage of the extracellular segment of the membranous cytokines or of their receptors (ADAM), etc. Examples of sequences cleaved by these proteases are given in Table 2. These intervening proteases play an important role in the evolution of the tumour or of the inflammatory tissue and in particular in the invasion of the surrounding tissues and the formation of metastases, or the invasion by cells specialised in inflammatory reaction. For example, the MMP rate in the tumoral or inflammatory environment is far higher than that present in a normal tissue because, in particular, control of the expression of these MMP depends upon the action of certain cytokines. It is therefore possible to take advantage of this differential expression in order to amplify the principle of targeting the anti-tumoral or anti-inflammatory compound by only allowing activation of this compound on the targeting sites. Simple prodrugs have already been designed but they do not resolve the problem of uniform distribution of the medication over the whole of the organism because there is no accumulation of the prodrug within the cancerous or inflammatory tissue. On the contrary, in this invention, one proposes a means of accumulating the anti-tumoral or anti-inflammatory principle only in the tumoral or inflammatory tissue already having cells in an apoptotic phase, and activating the medication essentially in this tissue. In other words, the C-L or L-C set forms a vectorisation-activation system for anti-tumoral or anti-inflammatory compounds. This set therefore corresponds to the following double imperative: reduce the average effective concentration of the medication in the organism, i.e. reduce the secondary toxic effects, and limit the action of the medication to the only interesting tissue, i.e. increase its effectiveness.

The cleavable linker L (or L0) is therefore a linker which is at least partially peptidic comprising a recognised sequence and cleaved by an intervening protease present by a majority in the targeted tissue. Linker L or L0 can therefore be represented as including two parts, L1-L2, designed such that the intervening proteases cleave the peptidic linkage of the linker between L1 and L2 and such that the released molecule L2-A or A-L1, according to the molecules selected, is a therapeutically active molecule, preferably at least as much as the initial molecule A. The length of parts L1 and L2 can be optimised as a function of the necessary accessibility to the active site of the intervening proteases, with the constraint of limiting as far as it can the final size of the final molecule for the reasons given above. In order to take into account all of the possibilities associated with the final structure of the therapeutic compound, A-L-C or C-L-A, the most general structure proposed for linker L is the following:
L=D-(L1-L2)ₙ (F5A)
L=(L1-L2)ₙ-D (F5B)
wherein D, L1, L2 and n are defined as above.

The bond between the different functional elements of the molecules of the invention can be implemented by any chemical, enzymatic or genetic coupling method known in its own right by the one skilled in the art. Thus, these can be chemical, peptidic, nucleic bonds, etc. The groups can be coupled to one another by maleimide, succinimide, intein, biotin, amine, amide, carboxylic, phosphate, ester, ether bonds, etc.

In a variation of the invention, the linker L is bonded to molecule C (and/or A) by a maleimide group, known for its fast and total reaction with a thiol group carried by a cysteine residue accessible to targeting segment C (and/or molecule A).

In another variation, the linker L is bonded to molecule C (and/or A) by the reaction of the terminal carboxylic group of peptide L with an amino group carried by the therapeutic segment A (or targeting segment C).

In another embodiment, linker segment L is coupled to the C-terminal end (in the case of a H₂N-L-A molecule) or the N-terminal end (in the case of an A-L-COOH molecule) of segment C by means of an intein. The benefit of this embodiment of the final molecule, relative to the previous embodiment using a bond by maleimide group, is that it maintains the possibility of providing a free cysteine in the molecule, for another functionalisation or for possible radioactive-labelling for following the medication. There is indeed a great therapeutic benefit in being able to follow and control, by means of imagery, the biodistribution and the kinetics of this biodistribution for this type of targeted medication.

In some embodiments, segment L (or the cleaving function) forms part of the C or A element. This can be for example an N-terminal or C-terminal extension of segment C or A, in particular when the latter is peptidic in nature.

The molecules of the invention can be assembled in one or more steps, according to the coupling techniques used. Thus, a C-L type molecule can be realised a first time, then coupled with one or more therapeutic molecules A. Alternatively, when the bonds between the functional segments call upon different chemical reactions, a simultaneous synthesis or assembly is possible.

Given the size of the linker segment (generally between about five and about twenty residues, for a segment which is peptidic in nature), the purely peptidic version of the linker L is preferably obtained by direct synthesis, using in particular the current peptide synthesizers and the classic synthesis methods, in particular in solid phase. The advantage of direct synthesis of the linker is that it allows the use of non-natural amino acid residues thus enabling better adaptation of the sequence to the intervening proteases. This recognition sequence can be further modified in relation to the natural recognition sequence by an enzyme or a cleaving factor, for example so as to provide it with better affinity or specificity to the protease in question.

The length of the linker segment can be adapted by the one skilled in the art as a function of the requirements and of the nature of segments C and A. In general, a fairly short and essentially non-immunogenic linker segment is used. This is typically a segment which is peptidic in nature comprising between 3 and 20 amino acid residues, preferably between 3 and 15, and even more preferably between 4 and 12. In some cases, as indicated below, it can be beneficial to modify the length of segment L, in particular so as to distance the peptidic part of segment C or A, or so as to create molecules with a particular function (penetration into the cell, action on neighbouring cells, etc).

Thus, there can be an advantage in linker L or L0 containing a spacing part in order to distance the cleaving site (and the peptidic part) from the targeting segment C. In this case, L or L0 can take on one of the two following structures:
L′1-L″1-L2 (F6A)
L1-L″2-L′2 (F6B)
wherein, in both cases:
L′1 is a non-peptidic spacing group and L″1 is the actual peptidic part;
L′2 is a non-peptidic spacing group and L″2 is the actual peptidic part.

The non-peptidic spacing group can be selected from any synthetic chemical molecule, preferably chemically stable and only slightly immunogenous. This can be in particular a polymer, for example of polyoxyethyelene, dextran, polyethyleneimine type, etc. A preferred example is polyoxyethylene, which is functionalised or not. In the above formulae, L″1-L2 and L1-L″2 can represent the same peptide. Moreover, the number of residues of peptidic segments L″1-L2 or L1-L″2 can vary between four and more than twenty, the optimal value being about six.

In a particular example of an embodiment, the cleavable linker segment corresponds to the following formula:
L (or L0)=—(CH₂—CH2-0)ₘ—(CH₂)ₙ—CO-L″1-L2 (F7A)
L (or L0)=L1-L″2-NH—(CH₂)ₙ—(OCH₂—CH₂)ₘ— (F7B)
wherein m is an integer greater than or equal to 0 and L″1 and L″2 are a peptide the sequence of which comprises at least one cleaving site for one of the proteases generally present in the targeted environment. It will be noted that L″1-L2 and L1-L″2 can represent the same peptide. The number of residues of peptidic segments L″1-L2 or L1-L″2 can vary between four and more than twenty, the optimal value being about six.

In the following examples a set of peptidic sequences for L″1-L2 or L1-L″2 are shown corresponding to the various criteria given in the whole of the above text:
J₋ₙ--J₀-B₁-B₂-J₃--J_{c} (S6)
wherein:
J₋ₙ--J₀-B₁ indifferently represents segments L″1 and L1;
B₂-J₃--J_{c} indifferently represents segments L2 and L″2;
peptidic bond B₁-B₂ is the bond cleaved by the intervening proteases.
n and c can vary between 0 and approximately 10 and depend upon the end chosen for binding to segment A; for the end linked to A, the value of n or c will be weak and preferably equal to 0 (J₃ only present) and for the opposite end the value of n or c is not limited and the corresponding residues will be chosen as a function of the specificity of the targeted intervening proteases.

The following table gives an example of all B₁-B₂ sequences recognised and cleaved by the different intervening proteases and which can be used preferably for a cleavable segment L:

TABLE 2

| | | Example of protease |
|---|---|---|
| B₁ | B₂ | in question |
| Val/Ala/Leu/Met | X | Neutrophil elastase |
| Leu/Tyr/Phe | X | Cathepsin G |
| Ala | Leu | Proteinase 3 (neutrophils) |
| Leu | Val | |
| Val | Cys | |
| Gly | Leu/Ile | Collagenases: MMP-1, |
| | | -2, -8, -9, -13 |
| Gly | Val | MMP2, MMP-9 |
| Gly/Ala/Asn/Glu/ | Hydrophobes, | MMP-3 |
| Gln/Pro/Arg/His/Asn | natural or not | |
| Polars: | Hydrophobes, | MMP-7 |
| Arg/Asp/Glu/Gln/Thr/Asn | natural or not | |
| Hydrophobe: Ala | | |
| Ala | Val | ADAM |
| Asn | Val | ADAM-17 (TACE) |
| Arg | Phe | |

Sequences J-₁-J₀ and J₃-J₄ which surround cleavage site B₁-B₂ intervene in the linker interactions with the protease in question and so with the enzymatic reaction speed of cutting the B₁-B₂ bond. Residues J-₁, J₀, J₃ and J₄ of S5 will advantageously be selected in the following sets:

J-₁=preferably polar residue

J₀=Gly, Ala, Leu, Ile, Val, Phe and any hydrophobic, non-natural amino acid residue.

J₃=Gly, Ala, Leu, Ile, Val, Phe

J₄=Gly, Ala, Leu, Ile, Val, Phe or any hydrophobic non-natural residue or absent.

The other residues, J-2--J-n and J5--Jc can be any natural or non-natural amino acid residue according to the requirements.

On the other hand, the length and/or the properties of the linker segment can be adjusted, for example in order to design a molecule enabling therapeutic compound A to interact with or to penetrate into a neighbouring cell of the target cell onto which segment C conveys it. In this case, for example, linker segment L:
must be sufficiently long to reach the neighbouring cells. For this type of linker, one preferably chooses a chemical oligomer, for example of polyoxyethylene type, comprising a sufficient number of monomers so that the length of the linker is between approximately 80 and 200 angstrom, typically between 130 and 150 angström; and/or
comprises a domain enabling or facilitating the passage of A in the cellular membrane and, should the occasion arise, a cleaving region sensitive to the intra-cellular enzymes.

In this embodiment, the linker advantageously has one of the two following structures:
L or L0=LE-LTM-L3-A (F9A)
L or L0=A-L3-LMT-LE (F9B)
wherein LE is an essentially extracellular part of the linker, LTM is a transmembranous part and L3 is a function or an element which can be cleaved by the intracellular proteases or esterases (e.g., cytosolic).

Part LE is preferably sufficiently hydrophilic so as to enable appropriate solvation in aqueous medium so as to obtain a sufficiently extended structure.

Part LTM, with a length at least equal to approximately 40 Å, is preferably sufficiently amphiphilic so that, on the one hand, its structure remains weakly compact in the extracellular aqueous medium and so that, on the other hand, it can cross the hydrophobic medium of the plasmic membrane.

The chemical composition of LE and LMT can for example be as follows:
LE=—[O—(CH2)ₚ-O]— (F10)
LMT=—[O—(CH2)_{q}-O]— (F11)
wherein q and p are integers different from 0, q>p such that, for LMT, the lipidic environment of the membrane is, from the energy point of view, more favourable than the aqueous environment. Values p=2 and 5≧q≧3 are preferable.

As indicated above, the targeting element can be synthesised by techniques known in their own right from chemistry or biology. The C-L element is moreover a particular object of the invention.

Therapeutic Segment A

The invention can be implemented with any type of therapeutic molecule likely to be associated with segment C. These can be chemical compounds, medications, small molecules, etc. of peptidic, nucleic, lipidic compounds, etc.

In general, therapeutic segment A is a molecule showing biological activity. Preferably, this biological activity is reduced (or non-existent) when the therapeutic segment is bonded to linker segment L and targeting segment C. For this reason, biological activity is expressed in particular in the environment of the pathological tissues, following in vivo targeting and cleaving.

Therapeutic compounds can have varied properties, in varied therapeutic domains. These can be medications which are already known, or new molecules or those being developed. They can be compounds the mode of action of which involves penetration into the cells or the action of which only involves interaction on the surface of the plasmic membrane. The preferred compounds A of the invention are anti-tumoral or anti-inflammatory compounds.

Anti-Tumoral Compounds

In a particular embodiment, the biologically active molecule shows anti-tumoral properties. A can be any anti-tumoral molecule or an active derivative of these same molecules. The only constraint is that these anti-tumoral compounds can be chemically bonded to the rest of the vectorisation molecule.

As described above, A can be released in the extra-cellular medium and diffused passively or actively within the neighbouring cells or else be conveyed into these cells by an LE-LMT type bond and then released by the action of endogenous proteases or esterases.

An example of an anti-tumoral compound is provided by the molecules of the family of antracyclins and their derivatives.

These antracyclin molecules contain an amine sugar. The amino group is advantageously used for binding to segment L (for example to part L2 or LMT) of the linker defined above.

Another example of an anti-tumoral compound is provided by the molecules of the family of TNFα or derivatives thereof. These molecules act upon surface receptors of cells and induce apoptosis.

Of the molecules from the family of TNFα, the TRAIL or Apo2L (P_W19777) factor is probably the most beneficial in so far as the normal cells seem protected from its action, whereas the tumoral cells are sensitive to them and can be selectively eliminated by the action of this pro-apoptotic cytokine. This factor can therefore be very useful in the so-called “solid” cancers, and this involves effective targeting so as to avoid as far as possible the secondary effects due to the presence of these molecules in the sanguineous medium. Like all of the molecules of the TNF family, TRAIL is initially a membranous protein associated in trimer and only the extracellular part is active. This extracellular part, TRAIL-Do, or a set containing TRAIL-Do, can therefore be targeted due to segment C and released into the inter-cellular space by means of the action of the intervening proteases on cleavable linker L which links C and TRAIL-Do. The therapeutic molecule formed in this way has the following structure:
C-L′1-L″1-L2-(TRAIL-Do) (according to formula F6A)
(TRAIL-Do)-L1-L″2-L′2-C (according to formula F6B)

An advantage of one or other of these arrangements is that the trimer assembly of the molecules of the TNF family, necessary for the binding to their receptors, is hindered by the presence of targeting and linker segments. Thus, the molecule remains weakly active as long as the linker is not cleaved by an intervening protease. So as to benefit from this particular advantage, the cleavable linker must have a sufficiently weak length, while maintaining appropriate accessibility to the intervening proteases.

Another example of cytokine, which is very beneficial for the treatment of certain cancers and in particular melanoma and intracranial glioma, is Interleukine-4 (IL4, 1310839). Unfortunately, given its important secondary effects, this protein can not be used without being appropriately targeted. Binding to a targeting segment C of human IL4 or of one of its isoforms or else of a set containing one of these proteins, by means of a cleavable segment makes it possible to form a therapeutic molecule which is beneficial in oncology:
C-L′1-L″1-L2-(IL4) (according to formula F6A)
(IL4)-L1-L″2-L′2-C (according to formula F6B)

Other examples of anti-tumoral compounds are in particular:
a) Methotrexate:

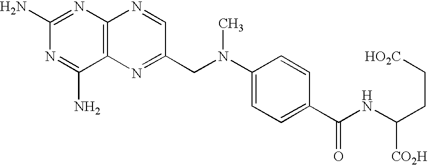

Methotrexate is an anti-tumoral compound currently used for the treatment of cancerous tumours. It is a folic acid analogue which first of all acts like a false substrate by inhibiting dehydrofolate reductase (DHFR). It also acts by indirect inhibition of thymidilate synthetase (TS).

Methotrexate contains an amino acid, glutamic acide, which can be inserted into the N-terminal end of a cleavable linker according to formula (F6B) or (F4B).

b) Methoxyestradiol

2-methoxyestradiol (1,3,5(10)-oestratriene-2,3,17β-triol 2-methyl ether) called 2ME₂, is a sub-product of the metabolism of oestrogens having the property of blocking the growth of endothelial cells with rapid division and of tumoral cells.

c) Taxanes

The effect of the molecules of this family is to block the cellular cycle in G2 and M by their action upon the microtubular cytoskeletons. This results in inhibition of the normal reorganisation necessary for implementation of the mitosis interphase.

Taxanes, and in particular docetaxel, include functions which can be used for modifications enabling them to be incorporated into the end of a peptide which can be cleaved by the intervening proteases of the tumoral environment. 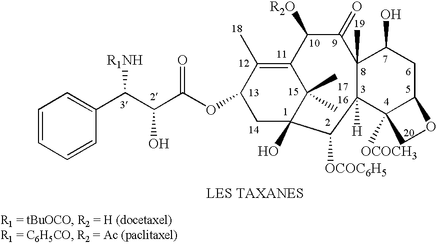
d) Modified Sugar Antipyrimidines:

Cytosine arabinoside (Ara-C) or cytarabine or Aracytine is the main representative of this family. Other molecules of this family are difluorodesoxycytidine (Gemcitabine).

e) Alkylating Agents:

Derivatives of “nitrogen mustards”, these agents have given birth to various anti-tumoral compounds acting on nucleic acids and so in intra-cellular space. These are in particular Melphalan and Chlorambucil, two bi-functional alkylating agents beneficial for the easy binding to a cleavage peptidic linker.

Melphalan is Phenylalanine Mustard (L-PAM) and is non-natural amino acid which can be easily introduced at the start or at the end of the peptidic synthesis of a linker cleavable by intervening proteases:

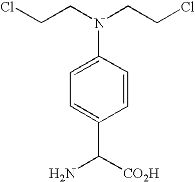

Chlorambucil only comprises one carboxylic function and can only be introduced directly into a peptide at the end of the synthesis of the peptidic linker cleavable by the intervening proteases and at the N-terminal end.

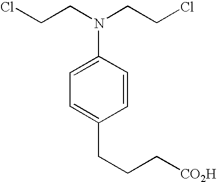

By using a bivalent bond segment such as ethanolamine or diaminoethane, this compound can also be introduced to the C-terminal end of the cleavable peptidic linker.

In both cases the molecule released into the tumoral environment is a hydrophobic amino acid derivative such as for example Leu-Melphalan or Chlorambucil-Leu. These compounds can easily cross the plasmic membrane of tumoral cells passively and act upon the nucleic acids, either directly because the alkylating function is not modified or after cleaving the additional amino acid by the endogenous peptidases or esterases.

Anti-Inflammatory Compounds

In another particular embodiment, the biologically active molecule shows anti-inflammatory properties.

Release of Peptides Derived from the N-Terminal Segment of Annexin I.

An example of these compounds is a peptide, here called NTA1, with anti-inflammatory properties identical to or derived from the N-terminal segment of annexin I. The anti-inflammatory properties of this peptide probably result from its action upon the inhibiting fMLP receptor thereby inhibiting chemotactism and activation of the phagocyte cells and in particular their degranulation and their production of toxic oxygen metabolites.

The sequence of the N-terminal segment of human annexin I is as follows:

| | |
|---|---|
| AMVSEFLKQAWFIENEEQEYVQTVKSSKGGP | (SEQ ID No 33) |

In a more general embodiment, the anti-inflammatory peptide derived from the N-terminal segment of annexin I will advantageously be selected from the following sequences:

(S7)

(SEQ ID No 34)

```
1 5 10 15 20 25 30
| | | | | | |
AMVSEFLKQAWFhaNpEQEYhpoVKooKGGP
            IN YYIE E DCVQTTQSSHVV
                    C LD Q IKSS TYS
                        M L NC CVP
                                          EA GG
```

The underlined sequence represents a so-called consensus sequence having the desired anti-inflammatory properties. Under each variable residue of this sequence is indicated a list of residues which can replace that indicated in the consensus sequence: a acid residue; h hydrophobic residue; p polar residue; o preferably Thr or Ser.

It is also possible to choose a shorter sequence for NTA1. One can thus delete the eight to thirteen first residues. In particular one can use the sequence:

| | | |
|---|---|---|
| (S8) | ENEEQEYVQTVKSSKGGP | (SEQ ID No 35) |

All of the mutations corresponding to this fragment and indicated for sequence (S7) will be usable in sequence S8.

In the inflammatory environment there is at least one protease likely to specifically cleave the NTA1 segment on one of the two Lysine 25 or 28 residues present in its N-terminal part -Thr-Val-Lys-Ser-Ser-Lys-Gly-Gly-. These proteases are normally responsible for the in vivo release of the N-terminal segment of annexin I.

In a first simple embodiment, the NTA1 peptide or a judicially mutated version is simply integrated into the N-terminal part of the C segment so as to form the therapeutic protein: NTA1-C. Segment C is taken here in its widest acceptance and defined above.

In a second embodiment, the NTA1 peptide or a judicially mutated version is linked to segment C by means of a single linker which can be cleaved by an intervening protease according to formula F6A or F6B or by means of a multiple linker comprising a branching element D as defined above (F5A, F5B).

In another embodiment, part A can also contain a repetition of one of the sequences chosen for the NTA1 segment so as to increase the local concentration of the anti-inflammatory peptide and so its effectiveness.
(NTA1)ₘ-C (F2)

In order to optimise the in vivo behaviour of the therapeutic molecule, m=2 is preferably chosen.

In another embodiment, it can be advantageous to bind the NTA1 segment to the C-terminal end of segment C. For this embodiment, one uses a bi-functional linker so as to link the C-terminal ends of C and NTA1 to one another:
C-L-NTA1
Release of Anti-Inflammatory Cytokines.

Chronic inflammatory diseases, and in particular rheumatoid polyarthritis, Chrohn's disease and Psoriasis, are provoked by a significant imbalance in the production in the cellular environment of a number of signalling molecules. The amplification and the perennisation of the inflammatory phenomenon in these diseases result from a complex balance between a large number of proteins with opposite influences, pro-inflammatory molecules and anti-inflammatory molecules.

Among the cytokines playing an anti-inflammatory role, interleukin 10 (IL10) (SwissProt, P22301), or any of its isoforms, is the most beneficial due to the regulating effect that it produces upon the inflammatory reaction. But like a lot of molecules which signal inflammatory reaction, IL10 has numerous functions including immune system stimulation functions. It is therefore very advantageous to target this protein at the actual inflammatory site such as to strictly localise its action.

IL10 acts as a homodimer on its hetero tetrameric receptor. The tri-dimensional structure of IL10 and of the complex with its receptor, IL10R, offers an additional advantage. Indeed, the structure of the IL10-IL10R complex, shows that the N and C-terminal ends of IL10 are relatively close in its structure. Moreover the N-terminal end is buried in the core of the receptor and the C-terminal end is located in the dimerisation region of this cytokine. For this reason, blockage by segment C-L or L-C of one of the N or C-terminal ends of IL10, prohibits formation of the complex and finally blocks its action. Activation of IL10 can therefore only be achieved by the action of the intervening proteases of the inflammatory environment, the effect of which is to release this cytokine exclusively in this environment.

The same reasoning can be applied to another anti-inflammatory cytokine, IL13 (P35225) or to one of its isoforms.

The therapeutic molecule based on IL10 or IL13 has one of the following general structures, according to the choice made for the positioning of IL10 or IL13:
C-L′1-L″1-L2-(IL10/IL13) (according to formula F6A)
(IL10/IL13)-L1-L″2-L′2-C (according to formula F6B)

The cleaving site of the intervening proteases is proposed between L″1 and L″1 or between L″2 and L′2. The length of residual segment L″1-L2 or L1-L″2 is such that it does not hinder formation of the IL10-IL10R or IL13-IL13Rα1/2 complex. It is easy to adjust the effective IL10 sequence so as to satisfy this constraint. In both cases segment L1 or L2 can be absent.

Release of Pro-Inflammatory Cytokine Inhibitors.

Therapeutic segment A can be selected from the non-activating inhibitors of the membranous receptors of the pro-inflammatory cytokines.

Natural inhibitors of certain cytokines exist and in particular interleukin 1 (IL1), a pro-inflammatory cytokine the incidence of which in inflammatory diseases comes immediately after that of TNFα which is the cytokine playing the central role. The soluble inhibitor of the IL1 receptor, IL1R, is a small protein, sIL1Ra (Swiss-Prot P18510), which acts by binding to IL1R without activating it, thus blocking IL1. The effectiveness of sIL1Ra was tested in various diseases, and in particular in rheumatoid arthritis and appears to be relatively active. However, the doses used in patients in subcutaneous injections are enormous, between 30 mg and 150 mg. The pharmacokinetics are unfavourable moreover because the half-life time in the circulation is only 21 mins, and this is very weak for therapeutic use in the case of rheumatoid arthritis.

As previously for IL10, the C-L-(IL1Ra) or (IL1Ra)-L-C set is totally inactive. Indeed, the structure of the IL1Ra-IL1R complex shows that the N and C-terminal ends of IL1Ra are very close in the structure and are buried in the core of the receptor. For this reason, the inhibitor can only be activated by the action of the intervening proteases of the inflammatory environment, the effect of which is to release the inhibitor exclusively in this environment.

The therapeutic molecule based on IL1Ra or on any of its isoforms, has one of the following general structures, according to the choice made for the positioning of IL1Ra:
C-L′1-L″1-L2-(IL1Ra) (according to formula F6A)
(IL1Ra)-L1-L″2-L′2-C (according to formula F6B)

The cleaving site of the intervening proteases is proposed between L″1 and L″1 or between L″2 and L′2. The length of the residual segment L″1-L2 or L1-L″2 is such that it does not hinder formation of the IL1-IL1Ra complex. As for IL10, it is easy to adjust the effective IL1Ra sequence in order to satisfy this constraint. In both cases, segment L1 or L2 can be absent.

Release of Anti-Inflammatory Medications.

Numerous non-peptidic molecules exist which have significant anti-inflammatory properties such as Glucocorticoids, non-steroid anti-inflammatories (NSAID), Methotrexate.

a) Glucocorticoids:

Initially, Glucocorticoids are natural hormonal molecules produced by the organism and intervene in numerous physiological processes. Their action is strictly intracellular and intervenes by their binding to nuclear receptors inducing the transcription of a certain number of genes, from where the complex role of these hormones with multiple inflammatory reaction steps comes. The use of Glucocorticoids and of their non-natural derivatives as medication is therefore still very delicate whereas their effectiveness can be very great. Their targeting and their strict release into the inflammatory environment are therefore crucial.

Glucocorticoids are steroid molecules, therefore fairly hydrophobic, and their action takes effect on the cell nucleus following passive diffusion through the plasmic membrane. For use as a targeted medication, Glucocorticoids can therefore simply be released into the inflammatory environment.

All of these molecules have chemical functions, for example hydroxyl groups, enabling them to be grafted onto peptidic molecules and in particular onto a segment L cleavable by intervening proteases as for anti-tumoral compounds.

The therapeutic molecule based upon Glucocorticoids has one of the following general structures, according to the choice made for the positioning of the molecule relative to the targeting segment:
C-L′1-L″1-L2-(O-Glucocorticoid) (according to formula F6A)
(Glucocorticoid-O)-L1-L″2-L′2-C (according to formula F6B)

In this form, these molecules are totally inactive.

The action of the intervening proteases releases either molecule L″1-L2-(O-Glucocorticoid) or molecule (Glucocorticoid-O)-L1-L″2, i.e. a prodrug which has to diffuse passively into the surrounding cells where they will be treated by the endogenous proteases and esterases in order to finally release the active molecule.

In this particular application of this invention, it is therefore important that the L″1-L2 or L1-L″2 segments are hydrophobic and as short as possible, taking into account the cleaving capacities of the intervening proteases. It is advantageous to limit to two the number of residues or even to one the number of residues in L″1 or L″2. It can also be advantageous to not introduce segment L1 or L2.

b) Non-Steroid Anti-Inflammatories:

Non-steroid anti-inflammatories form one of the most prescribed types of medication. These are for the most part cyclooxygenase inhibitors (COX-1 and COX-2), significant enzymes intervening in the arachidonic acid metabolism. These are medications generally reserved for the treatment of severe inflammatory diseases and used at doses which are generally very high and which therefore produce undesirable secondary effects. Among the NSAID, there is a type of compound with a carboxylic function which can be used for grafting them onto peptidic molecules and in particular onto a segment L which can be cleaved by intervening proteases as for the anti-tumoral or anti-inflammatory compounds mentioned above. Among these compounds, mention will be made of Aspirin, Olsalazine, Diclofenac, Etodolac, Sulindac, Idomethacin, Tenidap and all of the propionic acid derivatives such as Ibuprofen, Tiaprofenic acid, Naproxen, Ketoprofen and “profenides” in general. Other families of compounds can also be used, anthranilic acid and the like including the group of fenamates such as mefenamic acid, the group of derivatives of nicotinic acid such as niflumic acid.

Because all of these anti-inflammatory compounds are carboxylic acids, they must be fixed at the N-terminal end of the cleavable peptidic linker according to the following formulation:
(NSAID)-CONH-L″2-L′2-C

This configuration is advantageous in that it allows the introduction of the NSAID molecule directly at the end of the peptidic synthesis in solid phase of the L″2-L′2 segment. In this embodiment, segment L″2 must be sufficiently short and hydrophobic so as to enable the passive diffusion of the active (NSAID)-CONH-L″2 segment through the plasmic membrane of the target cells.

If, for particular reasons, it is necessary to use the C-terminal end according to formula: C-L′1-L″1-L2-(NSAID), segment L2 must in this case be bi-functional and hydrophobic as for example aminoethanol:
L2=H₂N—CH₂—CH₂—OH.
c) Methotrexate

Methotrexate also has anti-inflammatory properties and can be used in the same way as for its anti-tumoral properties described above.

Design of Inhibitors of Membranous Receptor of the Family of TNFR.

Strict homotrimerisation of the extracellular domains in the absence of the ligand is an important element of the correct function of the TNFR. This trimerisation of the empty receptor is essentially guaranteed by an N-terminal segment comprising the extracellular CRD1 domain. The protein-protein interactions are very specific and thus avoid the formation of heterotrimers due to the presence of different receptors on the surface of a same cell. One can take advantage of this property in order to produce new specific inhibitors from any membranous proteins of the family of TNFR.

These inhibitors use a peptide, called here PCRDX, comprising at least the CRD1 domain of any X TNFR in order to block the trimerisation thereof in a very specific way and so to block its function: by binding to a membranous sub-unit of the TNFR (monomer), the free PCRDX peptide, in the form of a monomer or dimer, blocks the trimerisation of this membranous sub-unit without activating the receptor because the latter can no longer acquire its trimeric structure.

In a simple embodiment, one can use a PCRDX peptide, or a mutated version of this domain, bonded to the C segment by an L1-L2 linker which can be cleaved by one of the intervening proteases generally present in the inflammatory environment such as only to release the inhibitor on the targeted inflammatory site. The general structure of the therapeutic molecule of this invention is therefore:
C-L1-L2-PCRDX or PCRDX-L1-L2-C
wherein cleaving is implemented between L1 and L2.

The C-L1-L2-PCRDX structure is directly active because it leaves the C-terminal end of the CRD1 free enabling it to be bonded to the CRD1 of a cellular receptor.

The PCRDX-L1-L2-C structure has the additional advantage of only making the CRD1 segment active when the latter is released by the cleaving of segment L1-L2, thus limiting the risk of secondary effects. Indeed, in this configuration, the C-terminal end of the PCRDX is encumbered by the presence of the L1-L2-C segment, unadapted to any interaction with an extracellular domain of a TNFR, thus making its interaction with the latter more difficult. Activity is covered by the cleaving of the L1-L2 linker.

The following list gives examples of minimum sequences which the various possible PCRDX segments usable as an inhibitor of the trimerisation of TNFR1 and TNFR2 must contain:

TNFR1 inhibitor

(SEQ ID No 36)

DSVCPQGKYI HPQNNSICCT KCHKGTYLYN DCPGPGQDTD
CRECESGSFT ASENHLRHCL SS
TNFR2 inhibitor

(SEQ ID No 37)

PGTCRLREYY DQTAQMCCSK CSPGQHAKVF CTKTSDTVCD
SCEDSTYTQL WNWVPECLSS

These sequences can be mutated, in particular in the region of interaction with the original TNFR1 and TNFR2 receptors, so as to increase their affinity with these receptors.

Similar sequences coming from any cytokine receptor of the family of TNF can be used in order to produce specific inhibitors of these receptors.

The cleavable L1-L2 linker which can be cleaved can advantageously be selected from the peptidic sequences recognised and cleaved by the specific proteases the role of which is to release the extracellular part of the membranous TNFR or the TNF membranous precursors. These proteases belong to the family of ADAM already mentioned. Among these proteases, one will choose in particular the ADAM-17 or TACE protease specific to the release of TNFα and TNFβ (LTβ) and consequently one will select the sequence of the L1-L2 segment such that it contains one of the following sequences:

L1*L2=SPLAQA*VRSSSR (SEQ ID No 38) or fragments thereof, PLAQA*VRSSS (SEQ ID No 39), LAQA*VRSS (SEQ ID No 40), AQA*VRS (SEQ ID No 41), QA*VR (SEQ ID No 42), or any combination of groups of sequences located on either side of the cleaving marked by an asterisk, for example. PLAQA*VRS (SEQ ID No 43) or AQA*VRSS (SEQ ID No 44), etc.

Uses of Targeting and Therapeutic Compound Release Molecules

The molecules of the invention can be designed so as to target different types of cells or pathological tissues, preferably in human. They can be used in order to prepare medications and/or in therapeutic treatment methods.

Thus, a particular object of the invention consists of a pharmaceutical composition having a chimera molecule as defined above.

Another objet of the invention concerns the use of a chimera molecule as defined above for preparing a medication. In a preferred embodiment, these medications are anti-tumoral or anti-inflammatory medications.

When the therapeutic compound A is an anti-inflammatory, the molecules according to the invention can be used for the preparation of medications intended for acute diseases such as asthma, hemorrhagic rectocolitis, Crohn's disease, septic shock, collagen diseases and arthritis.

When therapeutic compound A is an antineoplastic, the invention can be used for the treatment of different tumours, in particular solid or liquid or hematopoietic tumours, in particular cancers of the breast, lung, intestine, colon and rectum, brain, meninges, stomach, oesophagus, liver, pancreas, bladder, head and neck, the male and female reproductive organs, the skin, etc.

The pharmaceutical compositions of the invention can include any excipient or vehicle which is pharmaceutically acceptable, such as salt, solutes, etc. These can be saline, buffer, isotonic, water solutions, etc. The compositions can further include other active agents, used in combination, simultaneously, separately or spaced out over time.

Another object of the invention concerns the use of targeting molecules as defined above for locally supplying active principles to the area surrounding pathological tissues in subjects.

This invention further concerns methods of treatment for a disease in a subject including the administration of a molecule or of a composition as defined above. Preferably, the disease in question is a cancer or an inflammation. The treatment method can further include a preliminary step consisting of a treatment allowing to produce cells involved in an apoptosis process in the pathological tissue. It also concerns methods for locally supplying active principles to the area surrounding pathological tissues in subjects, including the administration of a molecule or of a composition as defined above.

Within the context of the invention, the term “treatment” means preventive, curative, palliative treatment, as well as the care of patients (reduction of suffering, reduction of the size of a tumour or of the progression of the disease, improvement of life span, deceleration of the progression of the disease, reduction of the inflammatory site), etc. Furthermore, the treatment can be implemented in combination with other agents or treatments (chemotherapy, radiotherapy, gene therapy, etc.). The treatments and medications of the invention are intended in particular for humans.

In order to implement the therapeutic methods defined above, the therapeutic compound can be used in different doses and according to different protocols. The administration can be implemented by any method known by the one skilled in the art, preferably by injection, typically via the intraperitoneal, intratumoral, intradermic, intracerebral, intravenous, intra-arterial or intramuscular route. The doses administered can be adapted by the specialist. Typically, between approximately 0.01 mg and 100 mg/kg are injected. Of course, injections can be repeated. The invention can be used in mammals, in particular in human beings.

### EXAMPLES

### Example 1

### Example of Design of L Segments which can be Cleaved for the Release of Anti-Tumoral and Anti-Inflammatory Compounds

If segment L is purely peptidic and only contains natural residues, it will preferably be integrated into segment C at its N- or C-terminal end by the classic methods of molecular biology. It can however be integrated if necessary into segment A if the latter is peptidic and obtained by molecular biology.

In a particular embodiment, it is advantageous to prepare segment L or the L-A set extemporaneously in reactive form for subsequent bonding to segment C. An interesting example is in the case where a thiol group introduced by a Cysteine residue is present in segment C, preferably far from the bonding site of C to the negatively charged membranes. This thiol group makes it possible, by means of a simple, fast and total chemical reaction, to bind the L-A set provided with the maleimide functional group.

The synthesis protocol is described in FIG. 1. Fragment L consists of a protected peptide bonded to a rink acid-labile resin via the FMOC strategy well known to experts in the field. The reactive fragment has the property of forming a covalent bond with a nucleophilic group—here the SH group of a Cysteine—of the C protein. The reactive group can be a bromoacetamide or, like here, and more advantageously, a maleimide group. The spacer group between the maleimide and the L peptide can be an alkyl, alkoxy or poly alkoxy group terminated by a carboxylic function for the coupling to L. In the example shown in FIG. 1, the therapeutic segment is an anti-tumoral compound of the antracyclin family, doxorubicin. In FIG. 1, AA represents any sequence of amino acids which can form a cleavable linker. The example described below corresponds to the sequence AA=Gly-Ser-Gly-Val-Leu.

Synthesis of Rink Acid Fmoc-Leu-Resin (1):

5 g of 100-2OO Mesh rink resin (approximately 0.35-0.80 mmole/g), Fmoc-Leu-OH (3.2 mmoles) and DCCI (3.37 mmoles) are stirred for 5 minutes between 0° C. and 5° C. in 50 ml of DCE, then 60 mg of DMAP (0.5 mmole) are added and after 20 more minutes at 5° C., 275 μl of N-methylmorpholine (2.5 mmoles) are added. The mixture is stirred for 4 hours. The filtered resin is washed with 40 ml (for 30 secs each time) with the following solvents: 3 times methanol, 3 times DCE, 3 times DMA. The hydroxyl groups of the resin which have not reacted are blocked with acetic anhydride (13.5 mmoles) in 3 ml of pyridine and 15 ml of DMA then the resin is washed as follows (40 ml each time): 2 times isopropanol, 3 times DMA, 2 times isopropanol, 6 times DCE, 2 times isopropanol, 3 times DMA, 3 times isopropanol. The resin is then dried in a vacuum and has a Fmoc content of approximately 0.35 mmole/g.

Synthesis of Rink Acid Fmoc-Gly-Ser(Trt)-Gly-Val-Leu-Resin (2):

Washing and Extraction of the Fmoc Group at Room Temperature:

1 g of rink acid Fmoc-Leu-Resin (0.35 mmole) is treated in the following way each time for 3 minutes with 20 ml of 1 time isopropanol, 4 times DMA, 6 times 20% piperidine in DMA, 2 times DMA, 1 time isopropanol, 4 times DMA.

Fmoc-AAₙ-OH Coupling:

DIEA (3.5 mmoles) in 10 ml of DMA is added to the resin. After the resin has swollen, the amino acids protected by the Fmoc (1.75 mmoles) and the HBTU (1.72 mmoles) in DMA are added. After 40 minutes the resin is rinsed with 4 times 40 ml of DMA.

Synthesis of Rink Acid H-Gly-Ser(Trt)-Gly-Val-Leu-Resin (3):

The free N-terminal form of the peptide protected and bonded to the resin is obtained by the cleaving and washing protocol identical to protocol (2)°.

Synthesis of the Reactive Compound (4):

To 1 g of resin (3) (3.5 mmoles in DMA) one adds 3.5 mmoles of DIEA, 1.75 mmoles of N-maleoyl-β-alanine and 1.72 mmoles of HBTU. After 2 hours, the resin is washed with 4 times 20 ml of DMA then dried in a vacuum.

Separation of the Protected Peptide of the Resin so as to Obtain (5):

The resin is dispersed in DCM and treated with 20 ml of AcOH/DCM (10/90 v/v) cleaving mixture for one hour, then filtered, washed 3 times with 20 ml of cleaving mixture then 3 times 20 ml of DMA in order to extract the peptide from the resin. Hexane (15 times the volume) is added to the filtrate so as to remove the acetic acid in azeotropic form. The resulting protected peptide is dried in a vacuum and purified by “flash” chromatography.

Coupling of the Protected Peptide to the Anti-Tumoral Doxorubicin Compound:

Protected from the light, one adds to the compound (5) (0.3 mmole in 2 ml of DMA), 3 mmoles of DIEA, 0.3 mmole of HBTU and 0.3 mmole of Doxorubicin. After 2 hours, the solvent is evaporated and the crude product is diluted in acetonitrile, purified by “flash” chromatography on silica gel.

Synthesis of the Final Compound (7):

Protected from the light, the compound (6) is diluted in a solution with 1% TFA and 5% triethylsilane in DCM. After 2 hours, the crude product is evaporated in a vacuum then dissolved in acetonitrile, purified with HPLC and lyophylised.

### Example 2

### Synthesis of a Mixed Peptidic and Non-Peptidic Linker for the Release of Anti-Tumoral Compounds

The non-peptidic part of the linker is introduced by means of the compound (12), obtained according to the diagram of FIG. 2, replacing N-maleoyl-β-alanine used in the previous synthesis (FIG. 1). The diagram of FIG. 2 describes a linker with a general structure and the following protocol describes the particular case m=1, n=2, o=1.

To a solution of 2-(2-aminoethoxy)-ethanol (47.55 mmol) (8) in 100 ml of dichloromethane, one adds drop by drop at 0° C. a solution of t-butylpyrocarbonate (47.55 mmol) in 50 ml of dichloromethane. This is allowed to come back to room temperature and after two hours, the reactional mixture is dry-evaporated. The desired carbamate (9) is obtained in the form of a colourless oil.

To a solution of PPH₃/DIAD (7.5 mmol) in 25 ml of freshly distilled THF is added, at 0° C., neopentylic alcohol (7.5 mmol), the above carbamate (9) (7.5 mmol) and maleimide (7.5 mmol). The reaction is then left at room temperature overnight. The reactional crude substance is dry-evaporated then flash chromatographed on silicon so as to give the expected product (10).

To a solution of the above carbamate (10) (6.5 mmol) in 40 ml of dichloromethane is added 30 ml of trifluoroacetic acid. The mixture is subjected to ultrasound for 10 minutes, then stirred at room temperature for 1 hour. The solvents are evaporated and the residue washed with chloroform (3×) and ether (4×). The product obtained (11) in the form of trifluoroacetate is used as in the following step.

To the trifluoroacetate salt of the above product (11) (6 mmol) in suspension in dichloromethane (30 mL), is added DIEA (12 mmol) at room temperature. After one hour, diglycolic anhydride (6.5 mmol) is added. After two hours, dry evaporation is implemented and the product (12) is purified by flash chromatography on silicon.

The compound (12) is then used like the N-maleoyl-β-alanine compound for the synthesis of a mixed cleavable linker according to the protocol described in FIG. 1 for compounds (5) and (6).

### Example 2b

### Synthesis of an Arm Comprising a Spacer Group and a Cleavable Peptidic Group and Coupling on the One Hand to a Targeting Molecule C and on the Other Hand to Doxorubicin

Compound (12) of FIG. 2 with n=2, M=1 and o=1 was coupled to the AAn=Gly-Gly-Ala-Leu peptide according to the method described above (Example 2). The molecule obtained was coupled to the doxorubicin according to the method described in the above example (FIG. 1) so as to obtain the following compound (12):

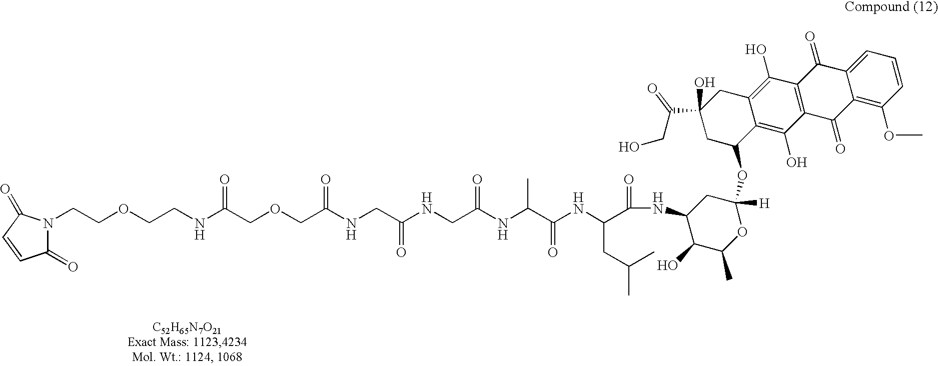

Compound 12 was then coupled to a targeting segment C-SH of family (S5) by means of the SH group which this comprises and according to the following general protocol: To 4.5 mg of lyophilised C-SH (1 equivalent) in 5.4 mL of PBS buffer (pH=6.7) degassed with nitrogen, a solution of 1 equivalent of to (12) (0.54 mg) in 540 μL of DMSO was added. The reaction was left to take place for 1 hour at room temperature, and then the reactional mixture was lyophilised.

The lyophylisate was returned to an H2O/DMSO 50/50 (2 mL) mixture and was purified with HPLC (Source 15RPC 10×250 mm column). The fraction corresponding to the final compound C-S-(12) was collected and then lyophilised.

The final compound C-S-(12) was subsequently subjected to the action of the different proteases (MMP2, MMP3 and MMP9) according to the following protocol:

A solution of C-S-(12) (60 μM) was prepared, i.e. 0.6 mg in 1 mL in a Tris-HCl 50 mM pH=7.5 buffer. This batch was separated into 3 aliquots, each of 330 μL. To each aliquot was added one of the MMP after activation (MMP2, MMP3 and MMP9), approximately 0.5 μg of each enzyme. This was left to incubate for 5 hours at room temperature. The aliquots were then purified with HPLC and the different absorbent peaks at 495 nm were analysed in ESI-TOF.

### Example 3

### Construction and Production of Anti-Inflammatory NTA1-C

Two constructions are proposed, of which one corresponds to the long version of NTA1 called NTA1l, according to sequence S7 (Seq ID 33), and the other to the short version, called NTA1c, according to sequence S8 (Seq ID 35).

NTA1c+:

(SEQ ID No 45)

5′ P-CGAAAACGAAGAACAGGAATACGTTCAGACCGTTAAATCTTCTAA
AGGTGGTCCGG-3′
NTA1c−:

(SEQ ID No 46)

5′ P-GATCCCGGACCACCTTTAGAAGATTTAACGGTCTGAACGTATTCC
TGTTCTTCGTTTTCGGGCC-3′
NTA1l+:

(SEQ ID No 47)

5′ P-CGCTATGGTTTCTGAATTCCTGAAACAGGCTTGGTTCATCGAAAA
CGAAGAACAGGAATACGTTCAGACCGTTAAATCTTCTAAAGGTGGTCCGG
-3′
NTA1l−:

(SEQ ID No 48)

5′ P-GATCCCGGACCACCTTTAGAAGATTTAACGGTCTGAACGTATTCC
TGTTCTTCGTTTTCGATGAACCAAGCCTGTTTCAGGAATTCAGAAACCAT
AGCGGGCC-3′
Ban II+:

(SEQ ID No 49)

5′ -GCGCTGTTAGCGGGTCCATTAAGTTCTGTC-3′
Ban II−:

(SEQ ID No 50)

5′ -GACAGAACTTAATGGACCCGCTAACAGCGC-3′

The used pGEX 6P1 plasmids are commercially available (Amersham biosciences), as are the enzymes (Biolabs) used: BamH I, EcoR I, Ban II, T4 DNA Ligase, CIP, T4 kinase.

Construction of NTA1c-C and NTA1l-C in pGEX 6P

The used expression vector is vector pGEX 6P1 (Amersham-Biosciences). This vector enables expression of the protein of interest fused to GST at its Nter end. The protein of interest is recovered without fusion protein after digestion by PreScission. The coding sequence of segment C (originating from vector pGEX2T, constructed at the laboratory) is inserted into this vector between the BamH I and EcoR I sites. The NTA1l or NTA1c segment is then inserted as cassettes into the pGEX 6P vector containing the coding sequence of segment C between sites Ban II and BamH I.

The pGEX 6P vector has two Ban II sites. The first step therefore consists of making this site unique so as to use it as a cloning site for the NTA1 segment. The Ban II site located in position 3890 is removed by a step of directed silent mutagenesis (Quick Change kit, Stratagene, oligos Ban II+ and Ban II−) following the supplier's recommendations. The “pGEX-6P-mut” plasmid is then obtained.

The coding sequence of segment C is extracted from the pGEX2T plasmid by enzymatic digestion using the restriction enzymes BamH I and EcoR I (Biolabs). Briefly, 20 μg of DNA is digested sequentially by 200 U of enzyme, at 37° C. overnight. After each digestion, the DNA is purified after migration over agarose gel using the “GFX PCR DNA and Gel Band Purification kit” (Amersham-Biosciences). 20 μg of “pGEX-6P-mut” plasmid is also digested in the same conditions by BamH I and EcoR I so as to obtain the vector in which the coding sequences are inserted. So as to avoid a recircularisation of the vector, the latter is dephosphorylated by an incubation for 2 hours at 37° C. in the presence of 2 U of CIP (Biolabs). The “pGEX-6P-mut” vector, open in BamH I/EcoR I and dephosphorylated, is then purified using the Amersham kit. Ligation of the coding sequence of segment C (10 insert moles for 1 vector mole) in the “pGEX-6P-mut” vector is then implemented by incubating for 2 hours at room temperature in the presence of 400 U of T4 DNA ligase (Biolabs). The pGEX-6P-mut plasmids containing the coding sequence of segment C are then obtained.

The “NTA1l” and “NTA1c” cassettes are obtained by hybridisation of 100 pmol of the complementary oligos (NTA1c+/NTA1c− and NTA1l+/NTA1l−) at 95° C. for 5 mins in a buffer Tris HCl 20 mM pH7.5; NaCl 300 mM; EDTA 1 mM. The 5′ ends of the cassettes are then phosphorylated by incubation at 37° C. for 2 hours in the presence of 50 U T4 polynucleotide kinase (Biolabs). The enzyme is deactivated by incubation at 65° C. for 20 mins.

The final step consists of digesting the pGEX-6P-mut plasmids, the coding sequence of segment C, by Ban II and BamH I in order to insert the cassettes. 20 μg of DNA is digested sequentially by 50 U of Ban II and 100 U of BamH I, at 37° C. overnight. After each digestion, the DNA is purified after migration on agarose gel using the Amersham kit. Dephosphorylation of the vectors is implemented by incubation at 37° C. for 1 hour in the presence of 10 U of CIP, in order to avoid their recircularisation during the ligation step. Ligation of the cassettes in the vectors is implemented as described above. Sequences NTA1c-C and NTA1l-C cloned in the pGEX-6P-mut vector are thus obtained.

After each construction, the DNA sequences are verified with the sequencing kit Big Dye Terminator, Perkin-Elmer Applied Biosystems, on a Perkin-Elmer Abiprism 310 sequencer, according to the supplier's protocol.

Expression of NTA1c-C and NTA1l-C in E. coli

Expression is implemented in an E. coli BL21 gold (Stratagene) strain at 30° C. The bacteria are placed in culture in a Luria Berthani (Gibco) medium containing 150 mg/L ampicillin. When the turbidity of the cultures reaches an optical density at 600 nm (DO₆₀₀) of 0.6, expression of the proteins is induced by adding 1 mM IPTG (Sigma) and kept for 4 hours. The bacteria are then centrifuged at 5,000 rpm (JLA1.8000, Beckman centrifuge) for 10 mins at 4° C. and resuspended in 20 mL of complete S buffer (20 mM Tris-HCl pH 7.6; 500 mM NaCl; 1 mM EDTA; 2% glycerol (v/v); 1% triton X100 (v/v)) supplemented with 0.1 mM of PMSF (Sigma) in ethanol; 0.5 mM of DTT (Sigma) and 50 mg of lysozyme (Sigma). After incubation for 1 hour at 4° C., the extracts are homogenised by 10 sonications of 1 min (amplitude 65%, 1 sec sonication, 1 sec rest) with 1 min rest between each sonication. The proteins present in the soluble fraction (supernatant) are then recovered by centrifugation at 20,000 g, for 45 mins at 4° C.

Purification of NTA1c-C and NTA1l-C on GSTrap Column

10 mL of the soluble fraction is collected and diluted in 20 mL of binding buffer (50 mM Tris-HCl pH7.5; 150 mM NaCl). The proteins are then purified by affinity chromatography on GSTrap Fast Flow column (Amersham-Biosciences). A column of 5 mL is prepared following the manufacturer's instructions. The protein sample (30 mL) is loaded onto the column and the latter is washed with 10 volumes of binding buffer. After additional washing with 10 volumes of cut buffer (50 mM Tris-HCl pH7.5; 150 mM NaCl; 1 mM EDTA; 1 mM DTT), the protein is incubated directly on the column with 100 U PreScission (Amersham-Biosciences) at 4° C. for 20 hours. The protein of interest without a fusion partner is then recovered by washing with 15 mL of cut buffer.

Purification of NTA1c-C and NTA1l-C by Gel Filtration

A HiLoad 26/60 Superdex 75 (300 mL) (Amersham-Biosciences) column is balanced using 2 volumes of buffer A (ammonium bicarbonate 150 mM pH 7.9). The protein resulting from purification by GSTrap is then injected, and its elution is implemented with 2 volumes of buffer A. The protein purified in this way is aliquoted, lyophilised and kept at 20° C.

### FIGURES

FIG. 1: Synthesis of the prodrug with AAₙ=Gly-Ser-Gly-Val-Leu as a substrate for MMP2, MMP3 and MMP9 and N-maleoyl-beta-alanine as a reactive binding segment.

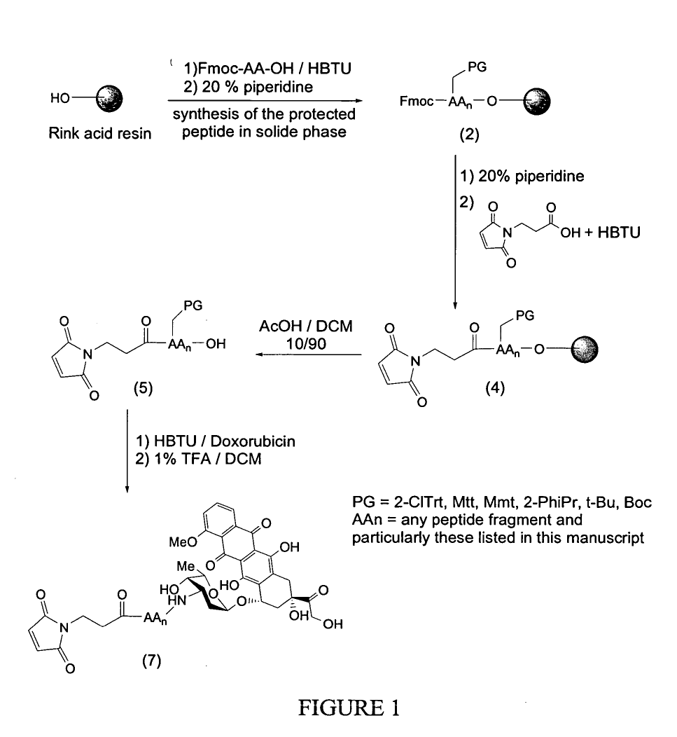

FIG. 2: Synthesis of the long non-peptidic linker replacing the short linker of N-maleoyl-beta-alanine used in the above protocol. Synthesis from 2-(2-aminoethoxy)-ethanol (8) (m=1, n=2, o=1)

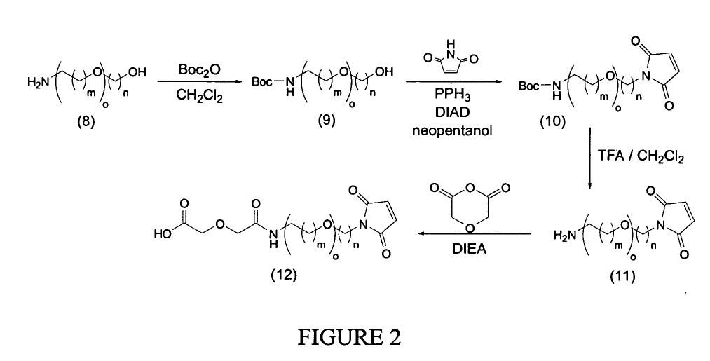

Abbreviations: AcOH: acetic acid; DCCI: N,N′-Dicyclohexylcarbodiimide; DCE: 1,2 Dichloroethane; DCM: Dichloromethane; DIEA: Diisopropylsilane; DMA: Dimethylacetamide; DMAP: 4-Dimethylaminopyridine; HBTU: 2-(1H-Benzotriazole-1-yl)-1,1,3,3-tetramethyluronium hexafluorophosphate; TFA: trifluoroacetic acid

1-18. (canceled)

19. A molecule comprising three segments
a targeting segment C capable of binding to membranes of cells engaged in an apoptosis process;
a therapeutic segment A comprising a biologically active compound; and
a linker segment L between the targeting segment and the therapeutic segment, said linker segment L being cleavable in vivo in the environment of a tissue or cell undergoing apoptosis.

20. The molecule according to claim 1, wherein said linker segment L comprises a chemical function recognised and cleaved by an enzyme or a set of enzymes specific to the environment of a tissue or of a cell in apoptosis.

21. The molecule according to claim 2, wherein said linker segment L comprises a sequence recognised and cleaved by a protease mainly present in the environment of a tissue or cell undergoing apoptosis, more particularly selected from the group consisting of a metalloprotease of the extracellular matrix, a urokinase, and a protease specific to cleavage of an extracellular segment of membranous cytokines or of their receptors.

22. The molecule according to claim 1, wherein said linker segment L comprises a sequence selected in that it contains at least one B1-B2 residue couple given in the following table:

| B₁ | B₂ |
|---|---|
| Val/Ala/Leu/Met | X |
| Leu/Tyr/Phe | X |
| Ala | Leu |
| Leu | Val |
| Val | Cys |
| Gly | Leu/Ile |
| Gly | Val |
| Ala | Val |
| Asn | Val |
| Arg | Phe |
| Gly/Ala/Asn/Glu/Gln/Pro/Arg/His/Asn | Hydrophobic, natural or not |
| Polar: Arg/Asp/Glu/Gln/Thr/Asn | Hydrophobic, natural or not |
| Hydrophobic: Ala | |

wherein X is any amino acid residue, natural or not.

23. The molecule according to claim 1, wherein said targeting segment C is capable of binding to membranes comprising lipids having a negative total electrostatic charge, in particular phosphatidylserine.

24. The molecule according to claim 5, wherein said targeting segment comprises the following peptidic sequence:
J¹-J²-J³-J⁴-J⁵-J⁶-Z⁷-U⁸-J⁹-J¹⁰-U¹¹-R-J¹³-J¹⁴-U¹⁵-K-G-X¹⁸-G-T-J²¹-E-J²³-J²⁴-U²⁵-J²⁶-J²⁷-J²⁸-U²⁹-J³⁰-J³¹-R-J³³-J³⁴-J³⁵-J³⁶-B³⁷-J³⁸-J³⁹-U⁴⁰-J⁴¹-J⁴²-J⁴³-U⁴⁴-J⁴⁵-J⁴⁶-J⁴⁷-J⁴⁸-J⁴⁹-R-J⁵¹-U⁵²-J⁵³-J⁵⁴-D-U⁵⁶-K-S-Z⁵⁹-L-J⁶¹-J⁶²-J⁶³-J⁶⁴-Z⁶⁵-J⁶⁶-J⁶⁷-U⁶⁸-J⁶⁹-J⁷⁰-J⁷¹-U⁷²-J⁷³-J⁷⁴-J⁷⁵-J⁷⁶
(S1)
wherein J, Z, U, X, and B represent amino acids such that:
J amino acids are selected, independently of one another, from natural amino acids, or from derivatives thereof, such that at least 50% of them are polar residues selected from R, N, D, C, Q, E, G, H, K, Orn, P, S, T and Y,
U amino acids are selected from A, C, G, I, L, M, F, W, Y, and V,
amino acid X¹⁸ is selected, independently of the other amino acids of the sequence, from A, N, C, Q, G, H, I, L, M, F, S, T, W, Y and V,
amino acid B³⁷ is selected, independently of the other amino acids of the sequence, from R, A, C, G, I, L, M, F, W, Y, and V,
amino acid Z⁷ is selected, independently of the other amino acids of the sequence, from D and E,
amino acids Z⁵⁹ and Z⁶⁵ are selected, independently, from E, D, K, and R,
and wherein exponents indicate the position of the amino acids in the sequence.

25. The molecule according to claim 6, wherein amino acids U and B are selected according to one of the examples given below:

| | U⁸ | U¹¹ | U¹⁵ | U²⁵ | U²⁹ | B³⁷ | U⁴⁰ | U⁴⁴ | U⁵² | U⁵⁶ | U⁶⁸ | U⁷² |
|---|---|---|---|---|---|---|---|---|---|---|---|---|
| Ex 1 | V | L | M | I | L | R | I | Y | L | L | V | L |
| Ex 2 | A | I | I | I | L | R | I | Y | L | L | I | L |
| Ex 3 | A | I | I | I | L | R | I | Y | L | L | M | V |
| Ex 4 | A | L | M | L | L | R | I | Y | L | L | I | M |
| Ex 5 | A | L | M | I | I | R | V | Y | L | L | I | M |
| Ex 6 | A | L | M | I | I | R | I | F | L | L | I | M |
| Ex 7 | A | L | M | I | V | R | I | F | L | L | I | F |
| Ex 8 | V | L | M | I | L | R | I | F | L | L | I | M |
| Ex 9 | A | L | M | I | L | R | I | F | L | L | I | M |
| Ex 10 | A | L | M | I | L | R | I | Y | L | L | A | A |
| Ex 11 | V | L | M | I | L | R | I | Y | L | L | V | L |
| Ex 12 | V | L | M | I | L | R | I | F | L | L | V | L |

26. The molecule according to claim 5, wherein said targeting segment C comprises a sequence selected from the group consisting of sequences SEQ ID Nos 23-32.

27. The molecule according to claim 1, wherein said targeting segment C comprises a sequence selected from the group consisting of all or part of an annexin, a C1 or C2 type domain of the blood coagulation factors, a domain V of a protein of the family of 2-Glycoproteins-I, a FYVE type domain, a PH type domain, and a fragment or a derivative thereof having at least 50% of identity.

28. The molecule according to claim 9, wherein said targeting segment C comprises a sequence selected from sequences SEQ ID Nos 1-16 and 17-22, preferably SEQ ID Nos 2-4, 6-8, 10-12, 14-16 and 19-22 or a fragment thereof.

29. The molecule according to claim 1, wherein said therapeutic segment A has anti-tumoral activity.

30. The molecule according to claim 11, wherein said therapeutic segment A is selected from the group consisting of a molecule of the family of TNFα or derivatives thereof (TRAIL-Do), a human IL4 molecule or one of its isoforms, a molecule of the family of anthracyclines or one of its active derivatives, preferably doxorubicin, a taxane molecule such as paclitaxel or docetaxel or one of its active derivatives, a methotrexate molecule or one of its active derivatives, 2-methoxyestradiol or one of its active derivatives, molecules of the family of antipyrimidines such as cytosine arabinoside or difluorodesoxycytidine or one of their active derivatives, and molecules of the family of alkylating agents derived from nitrogen mustards such as phenylalanine mustard (Melphalan) or a derivative such as Chlorambucyl.

31. The molecule according to claim 1, wherein said therapeutic segment A has anti-inflammatory activity.

32. The molecule according to claim 13, wherein said therapeutic segment A is selected from the group consisting of an N-terminal segment of human annexin I, in particular NTA1, anti-inflammatory cytokines, and in particular IL10 and IL13 or one of their appropriate mutants, non-activating inhibitors of membranous receptors of pro-inflammatory cytokines such as in particular an inhibitor of the IL1 receptor or an appropriate mutant of this inhibitor, glucocorticoids, non-steroid anti-inflammatories or their derivatives considered to be inhibitors of cylo-oxygenase enzymes 1 and 2, and Methotrexate, and an inhibitor of the membranous receptors of the family of TNFR, in particular peptides containing at least the corresponding CRD1 extracellular domain.

33. A pharmaceutical composition comprising a molecule according to claim 1.

34. A method for treating a disease in a subject, wherein the method comprises administering to the subject a pharmaceutical composition according to claim 15.

35. The method according to claim 16, wherein said disease is a cancer.

36. The method according to claim 16, wherein said disease is an inflammatory disease.


SEQUENCE LISTING (50 sequences)

SEQ ID NO: 1 - protein, 156 aa, Homo sapiens
DCRMPMGLSTGIISDSQIKASEFLGYWEPRLARLNNGGSYNAWSVEKLAAEFASKPWIQVDMQKEVIITGIQTQGAKHYLKSCYTTEFYVAYSSNQINWQIFKGNSTRNVMYFNGNSDASTIKENQFDPPIVARYIRISPTRAYNRPTLRLELQGC

SEQ ID NO: 2 - protein, 156 aa, Artificial Sequence
  Polypeptide built on the basis of  C1F5-S0
DCRMPLGMSTGIISDSQIKASEFLGYWEPRLARLNNGGSYNAWSVEKLAAEFASKPWLQIDMQKEVIITGIQTQGAKHYLKSCYTTEFYIAYSSNQINWQIFKGNSTRNVMYFNGNSDASTIKENQLDPPIVARYIRISPTRAYNRPTLRLELQGC

SEQ ID NO: 3 - protein, 156 aa, Artificial Sequence
  Polypeptide built on the basis of C1F5-S0
DCRMPMGLSTGIISDSQIKASEFLGYWWPRLARLNNGGSYNAWSVEKLAAEFASKPWIQVDLQKEVIITGIQTQGAKHYLKSCYVTEFYVAYSSNQINWQIFKYNSTRNVMYFNGNSDASTIKENQFDPPLVARYIRISPTRAYNRITLRLELQGC

SEQ ID NO: 4 - protein, 156 aa, Artificial Sequence
  Polypeptide built on the basis of  C1F5-S0
DCRMPMGLSTGIISDSQIKASEFLGYWEPRLARLNNGGSYNAWSVEKLAAEFASKPWLQIDLQKEVIITGIQTQGAKHYLKSCYTTEFYIAYSSNQINWQIFKGNSTRNVMYFNGNSDASTIKENQLDPPIVARYIRISPTRAYNRPTLRLELQGC

SEQ ID NO: 5 - protein, 150 aa, homo sapiens
KCQTPLGMASGHIRDFQITASGQYGQWAPKLARLHYSGSINAWSTKEPFSWIKVDLLAPMIIHGIKTQGARQKFSSLYISQFIIMYSLDGKKWQTYRGNSTGTLMVFFGNVDSSGIKHNIFNPPIIARYIRLHPTHYSIRSTLRMELMGC

SEQ ID NO: 6 - protein, 150 aa, Artificial Sequence
  Polypeptide built on the basis of   C1F8-S0
KCQTPMGLASGHIRDFQITASGQYGQWAPKLARLHYSGSINAWSTKEPFSWLKIDLLAPMIIHGIKTQGARQKFSSLYISQYIIMYSLDGKKWQTYRGNSTGTLMVFFGNVDSSGIKHNIFNPPIIARYIRLHPTHYSIRSTLRMELMGC

SEQ ID NO: 7 - protein, 150 aa, Artificial Sequence
  Polypeptide built on the basis of  C1F8-S0
KCQTPMGLASGHIRDFQITASGQYGQWAPKLARLHYSGSINAWSTKEPFSWIKVDLLAPMIIHGVKTQGARQKFSSLYISQFIIMYSLDGKKWQTYRYNSTGTLMVFFGNVDSSGIKHNIFNPPLIARYIRLHPTHYSIRSTLRMELMGC

SEQ ID NO: 8 - protein, 150 aa, Artificial Sequence
  Polypeptide built on the basis of  C1F8-S0
KCQTPLGMASGHIRDFQITASGQYGQWWPKLARLHYSGSINAWSTKEPFSWLKIDLLAPMIIHGIKTQGARQKFSSLYISQFIIMYSLDGKKWQTYRGNSTGTLMVFFGNVDSSGIKHNIFNPPLLARYIRLHPTHYSIRSTLRMEVMGC

SEQ ID NO: 9 - protein, 159 aa, homo sapiens
CSTPLGMENGKIENKQITASSFKKSWWGDYWEPFRARLNAQGRVNAWQAKANNNKQWLEIDLLKIKKITAIITQGCKSLSSEMYVKSYTIHYSEQGVEWKPYRLKSSMVDKIFEGNTNTKGHVKNFFNPPIISRFIRVIPKTWNQSITLRLELFGCDIY

SEQ ID NO: 10 - protein, 159 aa, Artificial Sequence
  Polypeptide built on the basis of  C2F5-S0
CSTPLGMENGKIENKQITASSFKKSWWGDYWEPFRARLNAQGRVNAWQPKANNNKQWLEVDLLKIKKITAVITQGCKSLSSEMYVKSFTIHYSEQGVEWKPFRLKSSMVDKINEGNTNTKGHVKNFPNPPRISRFIRVIPKTWNQSITLRLELFGCDIY

SEQ ID NO: 11 - protein, 159 aa, Artificial Sequence
  Polypeptide built on the basis of  C2F5-S0
CSTPLGIENGKIENKQITASSFKKSWWGDYWEPFRARLNAQGRVNAWQAKANNNKQWLEMDFLKIKKVTAVITQGCKSLSSEMYVKSFTIHYSEQGVEWKPYRLKSSMVDKIFEGNTNTKGHVKNFFNPPIISRFIRQIPKTWNQSITLRLELYGCDIY

SEQ ID NO: 12 - protein, 159 aa, Artificial Sequence
  Polypeptide built on the basis of  C2F5-S0
CSTPLGIENGKIENKQITASSFKKSWWGDYWEPFRLRLNAQGRVNAWQAKANNNKQWAEMDLLKIKKITAIITQGCKSLSSEMYVKSYTIHYSEQGVEWKPYRLKSSMVDKIFEGNTNTKGHVKNFFNPPIITRFIRVIPKTWNQSITIRLELFGCDIY

SEQ ID NO: 13 - protein, 153 aa, homo sapiens
CSMPLGMESKAISDAQITASSYFTNMFATWSPSKARLHLQGRSNAWRPQVNNPKEWLQVDFQKTMKVTGVTTQGVKSLLTSMYVKEFLISSSQDGHQWTLFFQNGKVKVFQGNQDSFTPVVNSLDPPLLTRYLRIHPQSWVHQIALRMEVLGC

SEQ ID NO: 14 - protein, 153 aa, Artificial Sequence
  Polypeptide built on the basis of C2F8-S0
CSMPLGMESKAISDAQITASSYFTNMFATWSPSKARLHLQGRSNAWRAQVNNPKEWLQIDLQKTMKITGITTQGVKSLLTSMYVKEYLISSSQDGHQWTLFYQNGKVKVFQGNQDSFTPVVNSLDPFLLTRYLRIHPVSWVHQIALRMEVLGC

SEQ ID NO: 15 - protein, 153 aa, Artificial Sequence
  Polypeptide built on the basis of  C2F8-S0
CSMPLGMESKAISDAQITASSYKTNMFATWSPSKARLHLQGRSNAWRAQVNNPKQWLQVDFQKTMKVTGVTTQGVKSLLTSMYVKEFLISSSQDGHQWTLFFQNGKVKVFQGFQDSFTPVVNSLDPPLLTIYLRIHPQSWVHQIALRMEVLEC

SEQ ID NO: 16 - protein, 153 aa, Artificial Sequence
  Polypeptide built on the basis of C2F8-S0
CSMPLGMESKAISDAQITASSYKTNMFATWSPSKARLHLQGRSNAWRPQVNNPKEWLQVDFQKTMKVTGVTTQGVKSLLTSMYVKEYLISSSQDGHQWTLFYQNGKVKVFQGNQDSFTPVVNSLDPFLLTRYLRIHPQSWVHQIALRMEVLEC

SEQ ID NO: 17 - protein, 86 aa, homo sapiens
TKASCKVPVKKATVVYQGERVKIQEKFKNGMLHGDKVSFFCKNKEKKCSYTEDAQCIDGTIEVPKCFKEHSSLAFWKTDASDVKPC

SEQ ID NO: 18 - protein, 86 aa, homo sapiens
  MISC_FEATURE (2)..(2): Xaa is Lys, Asp, or Glu
TXASCKXPXKXXTXXXXGERXXXQEKXXNGMLHGDKXSFXCXNXEXXCXYTEDXQCIDGTXEVPKCXXEHSXXXXXXTDASDVXPC

SEQ ID NO: 19 - protein, 86 aa, Artificial Sequence
  Polypeptide derived from domain 5 of beta2glycoprotein I
TEASCKVPVKRATVVYEGERVRIQEKFKNGMLHGDKVSFFCRNRERRCSYTEDAQCIDGTIEVPKCYREHSMLTWWRTDASDVKPC

SEQ ID NO: 20 - protein, 86 aa, Artificial Sequence
  Polypeptide derived from domain 5 of beta2glycoprotein I
TEASCKLPTKRMTVVYEGERVRIQEKFKNGMLHGDKISFFCRNRERRCSYTEDAQCIDGTIEVPKCYREHSMITWWRTDASDVKPC

SEQ ID NO: 21 - protein, 86 aa, Artificial Sequence
  Polypeptide derived from domain 5 of beta2glycoprotein I
TKASCKVPTKKMTVVYQGERVKIQEKFKNGMLHGDKISFFCKNKEKKCSYTEDAQCIDGTIEVPKCYKEHSSLAWWKTDASDVKPC

SEQ ID NO: 22 - protein, 86 aa, Artificial Sequence
  Polypeptide derived from domain 5 of beta2glycoprotein I
TKASCKVPTKKMTVVYQGERVKIQEKFKNGMLHGDKISFFCKNKEKKCSYTEDAQCIDGTIEVPKCYKEHSSLAFWKTDASDVKPC

SEQ ID NO: 23 - protein, 75 aa, Artificial Sequence
  sequence derived from a human annexine
GFDERADVETLRKAMKGLGTDEESILTLLTSRSNAQRQEISAAYKTLFGRDLLDDLKSELTGKFEKLVVALLKPS

SEQ ID NO: 24 - protein, 75 aa, Artificial Sequence
  sequence derived from a human annexine
NFDAERDALNIRKAIKGMGTDEDTIVQILTNRSNAQRQDIAFAYQRRTKRELASDLKSELSGHLERVILGLLKTS

SEQ ID NO: 25 - protein, 75 aa, Artificial Sequence
  sequence derived from a human annexine
DFSPSVDAEAIRKAIKGIGTDEDMLISILTERSNAQRQLIVKEYQAAYGRELKDDLKSELSGHFERLMVALVTPS

SEQ ID NO: 26 - protein, 75 aa, Artificial Sequence
  sequence derived from a human annexine
GFNAMEDVQTLRKAMKGLGTDEDALISVLAYRNTAQRQEIRTAYRSTIGRDLIDDLKSELSGNFERVIVGMLTPS

SEQ ID NO: 27 - protein, 75 aa, Artificial Sequence
  sequence derived from a human annexine
GFDPNQDAETLRTAMKGFGTDEEAILDIITSRSNRQRQEVSQSYKSLYGRDLIADLKSELTGKFERLIVGLMRPS

SEQ ID NO: 28 - protein, 75 aa, Artificial Sequence
  sequence derived from a human annexine
GFNPDQDAQALRKAMKGLGTDEDTIIDIIAHRSNVQRQEIRQAFKSHFGRELMTDLKSEISGDLERLILGLMMPS

SEQ ID NO: 29 - protein, 75 aa, Artificial Sequence
  sequence derived from a human annexine
PGDAIKDVEILRKAMKGFGTDEDAIVDIVANRSNDQRQKIKAAFKTSYGRDLIKDLKSELSGNLERLILALFMPS

SEQ ID NO: 30 - protein, 75 aa, Artificial Sequence
  sequence derived from a human annexine
HFNPDPDVAALRKAMKGIGTDEDAIIDILTSRSNTQRQEIAESFKAQFGRDLTEDLKSELSGKLERLIVALMYPS

SEQ ID NO: 31 - protein, 75 aa, Artificial Sequence
  sequence derived from a human annexine
GFDPLRDAEALRKAMKGFGTDEDAIIDLLGSRSNKQRQQILLSFKTAYGRDLIKDLKSELSGNFERTILALMKTS

SEQ ID NO: 32 - protein, 75 aa, Artificial Sequence
  sequence derived from a human annexine
GFDVDRDAKALRKAMKGMGTDEDAIIEILTSRTSDERQEIKQKYKATYGRELEEDLKSELSGNFEKVALALLDTS

SEQ ID NO: 33 - protein, 31 aa, homo sapiens
AMVSEFLKQAWFIENEEQEYVQTVKSSKGGP

SEQ ID NO: 34 - protein, 31 aa, Artificial Sequence
  peptide derived from the segment N-terminal of the annexine I
AMVSEFXXQAXXXXNXEQXXXXXXXXXXXXP

SEQ ID NO: 35 - protein, 18 aa, homo sapiens
ENEEQEYVQTVKSSKGGP

SEQ ID NO: 36 - protein, 62 aa, Artificial Sequence
  inhibitor of TNFR1 derived from CRD1
DSVCPQGKYIHPQNNSICCTKCHKGTYLYNDCPGPGQDTDCRECESGSFTASENHLRHCLSS

SEQ ID NO: 37 - protein, 60 aa, Artificial Sequence
  inhibitor of TNFR2 derived from CRD1
PGTCRLREYYDQTAQMCCSKCSPGQHAKVFCTKTSDTVCDSCEDSTYTQLWNWVPECLSS

SEQ ID NO: 38 - protein, 12 aa, Artificial Sequence
  cleavable bond
SPLAQAVRSSSR

SEQ ID NO: 39 - protein, 10 aa, Artificial Sequence
  cleavable bond
PLAQAVRSSS

SEQ ID NO: 40 - protein, 8 aa, Artificial Sequence
  cleavable bond
LAQAVRSS

SEQ ID NO: 41 - protein, 6 aa, Artificial Sequence
  cleavable bond
AQAVRS

SEQ ID NO: 42 - protein, 4 aa, Artificial Sequence
  cleavable bond
QAVR

SEQ ID NO: 43 - protein, 8 aa, Artificial Sequence
  cleavable bond
PLAQAVRS

SEQ ID NO: 44 - protein, 7 aa, Artificial Sequence
  cleavable bond
AQAVRSS

SEQ ID NO: 45 - DNA, 56 bp, Artificial Sequence
  oligonucleotide NTA1c+
cgaaaacgaagaacaggaatacgttcagaccgttaaatcttctaaaggtggtccgg

SEQ ID NO: 46 - DNA, 64 bp, Artificial Sequence
  oligonucleotide NTA1c-
gatcccggaccacctttagaagatttaacggtctgaacgtattcctgttcttcgttttcgggcc

SEQ ID NO: 47 - DNA, 95 bp, Artificial Sequence
  oligonucleotide NTA1l+
cgctatggtttctgaattcctgaaacaggcttggttcatcgaaaacgaagaacaggaatacgttcagaccgttaaatcttctaaaggtggtccgg

SEQ ID NO: 48 - DNA, 103 bp, Artificial Sequence
  oligonucleotide NTA1l-
gatcccggaccacctttagaagatttaacggtctgaacgtattcctgttcttcgttttcgatgaaccaagcctgtttcaggaattcagaaaccatagcgggcc

SEQ ID NO: 49 - DNA, 30 bp, Artificial Sequence
  oligonucleotide Ban II +
gcgctgttagcgggtccattaagttctgtc

SEQ ID NO: 50 - DNA, 30 bp, Artificial Sequence
  oligonucleotide Ban II -
gacagaacttaatggacccgctaacagcgc